(19) United States
(12) Patent Application Publication
(10) Pub. No.: US 2019/0223837 A1
(43) Pub. Date: Jul. 25, 2019

(54) DISPOSABLE PROBE

(71) Applicant: Neural Analytics, Inc., Los Angeles, CA (US)

(72) Inventors: Leo Petrossian, Los Angeles, CA (US); Robert Hamilton, Los Angeles, CA (US); Shankar Radhakrishnan, Los Angeles, CA (US); Jan Zwierstra, Los Angeles, CA (US); Matthew Hutter, Los Angeles, CA (US); Richard Castro, Los Angeles, CA (US); Seth Wilk, Los Angeles, CA (US); Roman Flores, Los Angeles, CA (US); Matthew Sylvester, Los Angeles, CA (US)

(21) Appl. No.: 16/255,531

(22) Filed: Jan. 23, 2019

Related U.S. Application Data
(60) Provisional application No. 62/620,940, filed on Jan. 23, 2018.
(60) Provisional application No. 62/688,874, filed on Jun. 22, 2018.

(51) Int. Cl. A61B 8/00; A61B 8/06; A61B 8/08

(52) U.S. Cl. CPC A61B 8/4477; A61B 8/06; A61B 8/4444; G01S 15/8988; A61B 8/0808; A61B 8/461; A61B 8/488

(57) ABSTRACT

Arrangements disclosed herein relate to a disposable probe system for use in an ultrasound system, the probe system includes a disposable probe configured to transmit or receive acoustic energy, and a base included in the ultrasound system. The disposable probe is configured to be removably attached to the base.

### CROSS-REFERENCE TO RELATED PATENT APPLICATIONS

This application claims priority from provisional U.S. Application No. 62/620,940, titled DISPOSABLE PROBE, filed Jan. 23, 2018, and provisional U.S. Application No. 62/688,874, titled DISPOSABLE PROBE, filed Jun. 22, 2018, which are both herein incorporated by reference in their entireties.

### BACKGROUND

A device (e.g., an ultrasound device, such as, a transcranial Doppler (TCD) device) may include a transducer or probe (e.g., a movable, robotic probe or a probe manually operated by a technician) configured to image a subject (e.g., a patient) or otherwise measure physiological data of a subject. The transducer may also be utilized to provide therapy to the subject. Traditionally, ultrasound measurement devices (e.g., a probe such as an ultrasound probe) are reused and need to be cleaned (e.g., disinfected) between each use (e.g., between subjects). However, as healthcare facilities increasingly focus on hygiene and infection control, utilization of the same probe for multiple subjects becomes more of a hygiene concern, for example, as probes or transducers often contact skin of subjects.

### SUMMARY

In some arrangements, a disposable probe system for use in an ultrasound system, includes a disposable probe configured to transmit or receive acoustic energy, and a base included in the ultrasound system, wherein the disposable probe is configured to be removably attached to the base.

In some arrangements, the disposable probe has a first connection interface. The base has a second connection interface. The disposable probe is configured to be removably attached to the base by having the first connection interface removably attach to the second connection interface.

In some arrangements, the first connection interface is configured to be attached to the second connection interface and detached from the second connection interface without using tools.

In some arrangements, the first connection interface includes a pin. The second connection interface includes a recessed contact configured to receive the pin. The first connection interface is configured to be attached to the second connection interface when the pin is received by the recessed contact.

In some arrangements, an electrical connection is closed when the pin is received by the recessed contact. The electrical connection is configured to allow transmission of data or power between the first connection interface and the second connection interface.

In some arrangements, the first connection interface includes a metal contact. The second connection interface includes a magnet configured to be coupled to the metal contact. The first connection interface is configured to be attached to the second connection interface when the magnet is magnetically attracted to the metal contact.

In some arrangements, the first connection interface is configured to be within a predetermined distance from a scanning surface. The disposable probe is configured to transmit or receive the acoustic energy with respect to the scanning surface.

In some arrangements, the disposable probe system further includes a robotic system. The base is configured to be coupled to the robotic system. The robotic system is configured to move the disposable probe by moving the base when the disposable probe is attached to the base.

In some arrangements, the disposable probe is configured to be manually operated.

In some arrangements, the disposable probe includes an ultrasound instrument configured to generate ultrasound energy.

In some arrangements, the ultrasound instrument includes at least one of a coupling layer, a first metallic layer, a lead zirconate titanate (PZT) layer, a second metallic layer, or a circuit board.

In some arrangements, the disposable probe includes a concave surface configured to focus the ultrasound energy.

In some arrangements, the disposable probe system further includes a membrane containing gel.

In some arrangements, the membrane is coated with a lubricant.

In some arrangements, the disposable probe system further includes a force sensor between the disposable probe and the base.

In some arrangements, the disposable probe includes a jack. The base includes a cavity. The disposable probe is configured to be attached to the base when the jack is inserted into the cavity.

In some arrangements, the jack includes at least one section. The base includes at least one electrical contact. Each of the at least one section is configured to electrically couple to a corresponding one of the at least one electrical contact when the jack is inserted into the cavity.

In some arrangements, the disposable probe includes at least one of a pressure sensor or a temperature sensor.

In some arrangements, the disposable probe includes one or more light emitting diodes (LEDs).

In some arrangements, the disposable probe includes one or more liquid-crystal displays (LCDs),

In some arrangements, the disposable probe is mechanically keyed to the base.

In some arrangements, the disposable probe includes a memory. The disposable probe is keyed to the base via at least one identifier stored in the memory.

In some arrangements, the disposable probe and the base form a unitary component when the disposable probe is removably attached to the base.

In some arrangements, a method for providing a disposable probe system for use in an ultrasound system includes providing a disposable probe configured to transmit or receive acoustic energy, and providing a base included in the ultrasound system, wherein providing the base includes configuring the disposable probe to be removably attached to the base.

### FIGURES

Features and aspects of arrangements will become more apparent from the following description and the accompanying example arrangements shown in the drawings, which are briefly described below.

FIG. 1 shows a cross-section view of a probe system according to various arrangements.

FIGS. 2A-2F show an example of a probe system according to various arrangements.

FIGS. 3A-3F show an example of a probe system according to various arrangements.

FIG. 4A shows an example implementation of the probe system in a system according to various arrangements.

FIG. 4B shows an example implementation of the probe system in a system according to various arrangements.

FIG. 4C shows an example implementation of the probe system in a system according to various arrangements.

FIG. 4D shows an example implementation of the probe system in a system according to various arrangements.

FIG. 5 shows an example implementation of a load cell of the probe system in a system according to various arrangements.

FIG. 6 shows an example implementation of a force transmitter according to various arrangements.

### DETAILED DESCRIPTION

The detailed description set forth below in connection with the appended drawings is intended as a description of various configurations and is not intended to represent the only configurations in which the concepts described herein may be practiced. The detailed description includes specific details for providing a thorough understanding of various concepts. However, it will be apparent to those skilled in the art that these concepts may be practiced without these specific details. In some instances, well-known structures and components are shown in block diagram form in order to avoid obscuring such concepts.

In the following description of various arrangements, reference is made to the accompanying drawings which form a part hereof and in which are shown, by way of illustration, specific arrangements in which the arrangements may be practiced. It is to be understood that other arrangements may be utilized, and structural changes may be made without departing from the scope of the various arrangements disclosed in the present disclosure.

Various arrangements disclosed herein relate to a probe system (e.g., a transducer system) that includes a disposable component and a base. The disposable component can releasably or removably attached to the base such that the disposable component is configured to be attached to and detached from the base by an operator without using tools. In some examples, the disposable component of the probe system can be the component that contacts, is proximate to, or otherwise interfaces with a subject. By replacing the disposable component for each use, hygienic concerns are mitigated as the probe system is used for multiple subjects (e.g., patients). Furthermore, the disposable component of the probe system can be manufactured using relatively inexpensive materials, thus making the disposable component relatively inexpensive and practical for one-time usage. The base can be made with relatively more sturdy materials given that the base is not replaced for each use. The base can be a part of or affixed to an ultrasound system, such as, a robotic ultrasound system configured to automatically move the probe or a manual ultrasound system that allows the probe to be moved by a human operator. Accordingly, in various arrangements, the probe system is safe, hygienic, and cost-effective.

In some arrangements, the probe systems as described herein can be used in conjunction with diagnostic ultrasound procedures (e.g., the ultrasound system including the base as described herein can be configured for the following procedures), such as, but not limited to, needle guidance, intravascular ultrasound (e.g., examination of vessels, blood flow characteristics, clot identification, emboli monitoring, and so on), echocardiograms, abdominal sonography (e.g., imaging of the pancreas, aorta, inferior vena cava, liver, gall bladder, bile ducts, kidneys, spleen, appendix, rectal area, and so on), gynecologic ultrasonography (e.g., examination of pelvic organs such as uterus, ovaries, Fallopian tubes, and so on), obstetrical sonography, otolaryngological sonography (e.g., imaging of the thyroid (such as for tumors and lesions), lymph nodes, salivary glands, and so on), neonatal sonography (e.g., assessment of intracerebral structural abnormalities through soft spots of a skull of an infant, bleeds, ventriculomegaly, hyrdrocephalus, anoxic insults, and so on), ophthamological procedures (e.g., A-scan ultrasound biometry, B-scan ultrasonography, and so on), pulmonological uses (e.g., endobronchial ultrasound (EBUS)), urological procedures (e.g., determination of an amount of fluid retained in a subject's bladder, imaging of pelvic organs (such as uterus, ovaries, urinary bladder, prostate, and testicles), and detection of kidney stones), scrotal sonography (e.g., to evaluate testicular pain, identify solid masses, and so on), musculoskeletal procedures (e.g., examination of tendons, muscles, nerves, ligaments, soft tissue masses, bone surfaces, and so on), bone fracture sonography, testing for myopathic disease, estimating lean body mass, proxy measures of muscle quality (e.g., tissue composition), nephrological procedures (e.g., renal ultrasonography), and the like.

In some arrangements, the probe systems as described herein can be used in conjunction with therapeutic ultrasound procedures (e.g., the ultrasound system including the base as described herein can be configured for the following procedures), such as, but not limited to, high-intensity focused ultrasound (HIFU), focused ultrasound surgery (FUS), Magnetic resonance-guided focused ultrasound (MRgFUS), lithotripsy (e.g., breaking up kidney stones, bezoars, gall stones, and the like), targeted ultrasound drug delivery, trans-dermal ultrasound drug delivery, ultrasound hemostasis, cancer therapy, ultrasound-assisted thrombolysis, dental hygiene (e.g., cleaning teeth), phacoemulsification, ablation (e.g., of tumors or other tissue), acoustic targeted drug delivery (ATDD), trigger release of drugs (e.g., anti-cancer drugs), ultrasound-guided treatments (sclerotherapy, endovenous laser treatment, liposuction, and so on), and the like. In some arrangements, ultrasound is used for physical therapy applications, including, but not limited to, stimulating tissue beneath the skin's surface (e.g., by using very high frequency sound waves, such as, as an example, between about 800,000 Hz and 2,000,000 Hz), treating musculoskeletal ailments with ultrasound exposure (e.g., ligament sprains, muscle strains, tendonitis, joint inflammation, plantar fasciitis, metatarsalgia, facet irritation, impingement syndrome, bursitis, rheumatoid arthritis, osteoarthritis, and scar tissue adhesion), and the like.

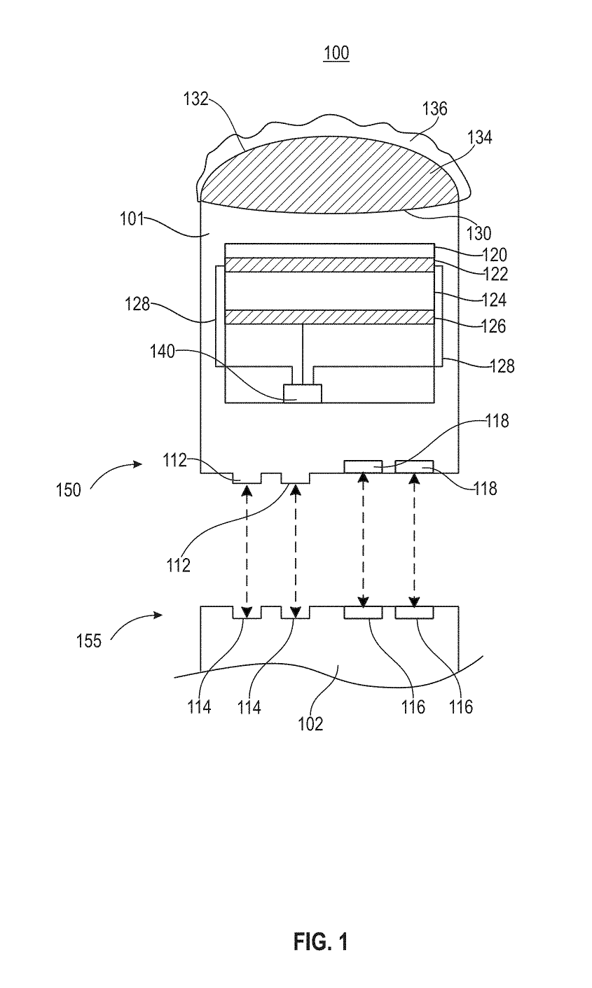

FIG. 1 shows a cross-section view of a probe system 100 according to various arrangements. The probe system 100 includes a disposable probe (or a disposable component of the probe) 101 and a base 102. In some arrangements, the disposable probe 101 is configured to be one or more of a camera, an ultrasound instrument (e.g., an ultrasound imaging instrument such as, but not limited to, transcranial Doppler (TCD), transcranial color Doppler (TCCD), a transducer, and so on), a thermal instrument, a near-infrared instrument (e.g., near-infrared spectroscopy), an optical camera instrument, a lighting instrument (e.g., for illumination), a spectrometer instrument, a microphone instrument, an electroencephalography instrument, an electromagnetic instrument, and so on. While an ultrasound instrument is used as an example of the disposable probe 101, the disposable probe 101 can be another device listed herein. The disposable probe 101 may include a single sensor/instrument or an array (e.g., a linear array, a circular array, and so on) of sensors/instruments.

In some arrangements, the base 102 is part of a system that is configured to receive the disposable probe 101. In particular arrangements, the base 102 is a part of an ultrasound system. For example, the ultrasound system can include a manually-operated system for positioning the disposable probe 101. For example, an operator can manually maneuver the disposable probe 101 during use (e.g., in collecting ultrasound data in the diagnostic uses mentioned above or providing therapy in the therapeutic uses mentioned above). In some arrangements, the manually-operated system is an ultrasound system that is configured to receive and connect with the disposable probe 101 (e.g., the manually-operated system includes the base 102 or has the base 102 attached thereto).

In some arrangements, the disposable probe 101 is configured to collect data (e.g., ultrasound data, sound data, light data, electromagnetic data, temperature data, pressure data, and so on) with respect to a subject and transmit signals corresponding to the data to the base 102 in the manner described herein. A system including the base 102 is configured to receive (e.g., by the base 102) the signals from the disposable probe 101 via a suitable wired or wireless connection. The system is configured to process, store, and/or display the signals.

In other arrangements, the base 102 is part of a robotic system (not shown) for positioning the disposable probe 101. In that regard, examples of the base 102 include but are not limited to, a TCD system. In some examples, the base 102 includes or is operatively coupled to positioning components such as but not limited to, the robotic system configured to control positions and operations (e.g., data collection) of the disposable probe 101. For example, the robotics are configured to translate the probe system 100 (e.g., the base 102 and the disposable probe 101 when the disposable probe 101 is attached to the base 102) along a surface of the skin of the subject and to move the probe system 100 toward and away from the head. In that regard, the robotic system is configured to move the disposable probe 101 indirectly by moving the base 102 when the disposable probe 101 is attached to the base 102 in the manner described. In some arrangements, an end (e.g., a bottom end) of the base 102 interfaces with or otherwise includes the robotic system. The robotic system includes components such as but not limited to, a motor assembly and so on for controlling the probe system 100 (e.g., control z-axis pressure, normal alignment, or the like of the probe system 100). In some arrangements, the registration of the probe system 100 against the subject is accomplished using the robotic system to properly position and align the probe system 100 (e.g., the disposable probe 101) with anatomic locations/areas of the subject.

In some arrangements, the bottom end of the base 102 is coupled to the robotic system. In some arrangements, the bottom end of the base 102 includes a threaded section along a portion of the body of the base 102, and the bottom end is configured to be secured in the robotic system via the threads (e.g., by being screwed into the robotic system). In other arrangements, the base 102 is secured in the robotic system by any other suitable connecting mechanisms such as but not limited to, welding, adhesive, one or more hooks and latches, one or more separate screws, press fittings, or the like. In some arrangements, the base 102 is a part of the robotic system (e.g., an integrated portion of the robotic system).

In some arrangements, the disposable probe 101 is releasably attached to the base 102. That is, the disposable probe 101 can be removably attached to the base 102 such that the disposable probe 101 can be attached to and detached from the base 102 by an operator without using tools. For example, the disposable probe 101 can include one or more leads or pins (e.g., pins 112) on or protruding from a bottom surface or a bottom end of the disposable probe 101. The pins 112 are configured to be received by or mate with corresponding recessed contacts 114 on a top surface or a top end of the base 102. The top end of the base 102 is an end opposite to the bottom end of the base 102. When the pins 112 are received by the recessed contacts 114, the pins 112 and the recessed contacts 114 are mated such that the bottom end of the disposable probe 101 is attached to the top end of the base 102 (e.g., the disposable probe 101 and the base 102 are structurally connected). In some examples, at least a portion of the bottom surface of the disposable probe 101 contacts at least a portion of the top surface of the base 102 when the pins 112 are received by the recessed contacts 114.

In some arrangements, in addition to providing structural support, the pins 112 and the recessed contacts 114 being connected (e.g., when the pins 112 are received by the recessed contacts 114) closes electrical connections between the disposable probe 101 and the base 102 to allow transmission of data (e.g., signals, information, and so on) and/or power between the disposable probe 101 and the base 102. That is, each of the pins 112 and a corresponding one of the recessed contacts 114 are configured to contact each other when each of the pins 112 is received by the corresponding one of the recessed contacts 114. For example, a first one of the pins 112 and a corresponding one of the recessed contacts 114 contacting one another or otherwise being electrically connected allows transmission of data from the disposable probe 101 and the base 102, and a second one of the pins 112 and a corresponding one of the recessed contacts 114 contacting one another or otherwise being electrically connected allows transmission of power from the base 102 to the disposable probe 101.

While FIG. 1 shows that the disposable probe 101 includes the pins 112 and the base 102 includes the recessed contacts 114, pins (such as but not limited to, the pins 112) can be disposed on either the disposable probe 101 or the base 102, or both of the disposable probe 101 and the base 102. Recessed contacts (such as but not limited to, the recessed contacts 114) can be disposed on either the disposable probe 101 or the base 102, or both of the disposable probe 101 and the base 102.

In some examples, the disposable probe 101 includes a battery or a wireless power interface (e.g., an inductive charging power interface) such that the pins 112 and the recessed contacts 114 are not used for transferring power between the disposable probe 101 and the recessed contacts 114. For example, the battery may include sufficient power for the functionalities of the disposable probe 101 as described herein such that additional power from the base 102 is not needed. In other words, the battery may include sufficient power for a single use of the disposable probe 101. In another example, the base 102 may include a wireless power generator (e.g., an inductive charging power interface) wirelessly coupled to the wireless power interface of the disposable probe 101 such that the base 102 can transfer wireless electrical signals to power the disposable probe 101 for the functionalities of the disposable probe 101 as described herein. In some arrangement, data is wirelessly transmitted and/or received between the ultrasound system including the base 102 (e.g., in addition to the wireless power supply such that the system is completely wireless).

In some examples, the pins 112 and the recessed contacts 114 are made from a metal material or a metal alloy material. In some arrangements, the recessed contacts 114 are made of a material (e.g., gold or titanium coating) that does not corrode over time and that allows for numerous uses for replacement of numerous disposable probes (each of which may be a probe such as but not limited to, the disposable probe 110) that can be attached thereto. In some arrangements, the locations of the pins 112 and the locations of the corresponding recessed contacts 114 are configured to direct an operator to properly align and orient the disposable probe 101 with respect to the base 102 during installation. For example, geometric distribution of the pins 112 and corresponding geometric distribution of the recessed contacts 114 form mirrored geometric shapes (e.g., triangles, squares, stars, and so on) such that the pins 112 can be received by the recessed contacts 114 only when the disposable probe 101 is in a particular orientation with respect to the base 101.

In some arrangements, the base 102 and/or the disposable probe 101 include magnets and in some examples, metal disks to further aid in installation and alignment of the disposable probe 101 with respect to the base 102. As shown, the base 102 includes magnets 116, while the disposable probe 101 includes metal contacts 118 configured to align with the magnets 116 (or vice versa) via magnetic attraction or coupling. That is, the magnets 116 are configured to be magnetically attracted to the metal contacts 118 due to magnetism. Thus, the base 102 is attached to the disposable probe 101, vice versa, and the base 102 and the disposable probe 101 can be held in place more securely during attachment because of the magnetic coupling. In other arrangements, the probe system 100 may include any other suitable mechanism (such as but not limited to, press fitting, compression fitting, snap fitting, friction fitting, screws, rivets, bolts, zippers, buttons, nails, hooks, elastic bands, latches, hook-and-loop fastener, ball joints, threaded sections, welding, adhesives, or another suitable fastener) in addition to the pins 112 and the recessed contacts 114 for securely fastening the disposable probe 101 to the base 102, and vice versa. In the arrangements in which the fastener is made of a conductive material (e.g., metal), the fastening mechanism between the base 102 and the disposable probe 101 can serve as an additional electrical connection between the base 102 and the disposable probe 101. In some arrangements, the disposable probe 101 is attached to the base 120, used on a subject, and then detached and disposed of, such that another disposable probe (such as but not limited to, the disposable probe 101) can be attached to the base 102 for use with another subject.

While FIG. 1 shows that the disposable probe 101 includes the metal contacts 118 and the base 102 includes the magnets 116, metal contacts (such as but not limited to, the metal contacts 118) can be disposed on either the disposable probe 101 or the base 102, or both of the disposable probe 101 and the base 102. Magnets (such as but not limited to, the magnets 116) can be disposed on either the disposable probe 101 or the base 102, or both of the disposable probe 101 and the base 102.

In other words, the disposable probe 101 includes a connection interface 150 configured to removably attach to a connection interface 155 of the base 102. The connection interface 150 includes one or more of the pins 112, the metal contacts 118, and other suitable connection mechanisms. In some examples, the connection interface 150 (e.g., a surface on which the pins 112 and the contacts 118 are disposed) is configured to be within a predetermined distance (e.g., 3 inches, 4 inches, 6 inches, 12 inches, and so on) from a scanning surface (e.g., a skin of a subject) during operations of the disposable probe 101 (e.g., when the connection interface 150 and the connection interface 155 are removably attached). The connection interface 155 includes one or more of the recessed contacts 114, the magnets 116, and other suitable connection mechanisms. In some examples, the connection interface 155 (e.g., a surface on which the recessed contacts 114, the magnets 116 are disposed) is configured to be within the predetermined distance (e.g., 6 inches) from a scanning surface (e.g., a skin of a subject) during operations of the disposable probe 101 (e.g., when the connection interface 150 and the connection interface 155 are removably attached).

When the disposable probe 101 is removably attached to the base 102, the length of the probe system 100 (including the length of the disposable probe 101 and the length of the base 102) is less than a predetermined length (e.g., 3 inches, 5 inches, 7 inches, 8 inches, 10 inches, 12 inches, and so on). The location of the connection interface 150 or 155 is within a predetermined distance (e.g., 0.5 inch, 1 inch, 1.5 inches, 2 inches, 3 inches, and so on) from a tip of the membrane 132 or the concave surface 130 (or a convex surface described herein). The location of the connection interface 150 or 155 is within a predetermined distance (e.g., 1 inch, 1.5 inches, 2 inches, 3 inches, 4 inches, and so on) from an end of the base 102 that is opposite to the end of the base 102 on which the connection interface 155 is located. In some examples, the location of the connection interface 150 or 155 is within a predetermined distance (e.g., 0.5 inch, 1 inch, 1.5 inches, 2 inches, 3 inches, and so on) from both the tip of the membrane 132 (or the concave surface 130 or a convex surface described herein) and the end of the base 102 that is opposite to the end of the base 102 on which the connection interface 155 is located. That is, the location of the connection interface 150 or 155 is equidistant from the tip of the membrane 132 (or the concave surface 130 or a convex surface described herein) and the end of the base 102 that is opposite to the end of the base 102 on which the connection interface 155 is located. In some examples, the location of the connection interface 150 or 155 is closer to the tip of the membrane 132 (or the concave surface 130 or a convex surface described herein) than to the end of the base 102 that is opposite to the end of the base 102 on which the connection interface 155 is located.

In some arrangements, the disposable probe 101 includes electronic components. For example, the electronics are configured to generate and/or collect energy such as but not limited to, ultrasound waves, infrared or near-infrared waves, x-rays, electromagnetic waves, thermal energy, optical energy, light, audio waves, electroencephalography energy, and so on.

In the example as shown in FIG. 1, the disposable probe 101 includes electronic components (e.g., a coupling layer 120, a first metallic layer 122, a lead zirconate titanate (PZT) layer 124, and a second metallic layer 126) configured to generate ultrasound energy. The coupling layer 120 is a matching layer that converts PZT impedance to air impedance (e.g., for transmitting ultrasound energy through the disposable probe 101). In some arrangements, the PZT layer 124 vibrates and has one or more wires 128 extending between the PZT layer 124 and a circuit board 140 (e.g., via the first and second metallic layers 122 and 126). In some arrangements, the first and second metallic layers 122 and 126 are made from silver. In other arrangements, the first and second metallic layers 122 and 126 are made from copper (e.g., for cost-effective considerations). One or more of the layers 120-126 and the circuit board 140 can be powered by one or more of a power connection provided by the pins 112 and the recessed contacts 114, by a wireless power connection, or by a battery in the disposable probe 101 in the manner described.

While the layers 120-126 are shown in FIG. 1, any suitable configuration of electronic components can be included within the disposable probe 101 to allow energy waves (e.g., ultrasound waves) to emanate from the disposable probe 101. In further arrangements, because the disposable probe 101 is disposable, relatively inexpensive electronic components can be used therein.

In some arrangements, a top end of the disposable probe 101 has a concave surface 130 (e.g., a concave lens) configured for focusing an ultrasound beam or ultrasound energy. As shown, the concave surface is located in the top end of the disposable probe 101, where the top end is an end opposite to the bottom end of the disposable probe 101. The concave surface 130 is configured to be adjacent to or to contact a scanning surface (e.g., the skin of the subject) in some examples. The concave surface 130 is configured with a particular pitch to focus generated energy towards the scanning surface. In some arrangements, the probe system 100 is a part of a TCD apparatus such that the top end of the disposable probe 101 is configured to be adjacent to or to contact and align along a human head (e.g., a side of the human head), and the concave surface 130 is configured to direct ultrasound wave emissions into the human head (e.g., towards the brain).

Air pockets between the disposable probe 101 (e.g., the concave surface 130) and the scanning surface (e.g., human skin) contribute to poor ultrasound transmission and signals. Accordingly, in some arrangements, to reduce or eliminate air pockets, a membrane 132 including gel 134 therein can be attached to the top end of the disposable probe 101, such that the gel 134 can be between the concave surface 130 and the scanning surface during operations of the probe system 100. That is, the membrane 132 and the gel 134 immerse the top end of the disposable probe 101 in the gel 134 to minimize or eliminate any air pockets therein. In some arrangements, the membrane 132 secures and restrains the gel 134 from leaking beyond the top end of the disposable probe 101. The gel 134 is contained between the membrane and the concave surface. The gel 134 can be any suitable ultrasound transmissive gel to improve ultrasound transmission.

In some arrangements, the membrane 132 is made from a malleable or compliant material to allow the membrane 132 to conform to the scanning surface at which the disposable probe 101 is placed for transmitting energy. The membrane 132 can be made from a suitable bio-compatible material. Furthermore, the membrane 132 is made from any suitably strong material to maintain the gel 134 within the membrane 132 as the disposable probe 101 moves along the scanning surface. The membrane 132 can be made from a material such as but not limited to, polyethylene (PE), polypropylene (PP), polycarbonate (PC), polyurethane (PU), polyetherimide (PEI), polyvinyl chloride (PVC), and polyether ether ketone (PEEK), a thin layer of silicone, biocompatible waterproof fibers or fabric, a medical curtain (e.g., a PE curtain), paper (e.g., treated paper), Tyvek®, leather (e.g., synthetic or genuine leather), and so on.

In some arrangements, the membrane 132 has a lens, balloon, or bubble shape. In some arrangements, the membrane 132 is coated with a lubricant 136 to provide better movement of the disposable probe 101 when scanning along the scanning surface. In some arrangements, the disposable probe 101 is included in a packaging separate from a packaging in which the membrane 132 is placed. In its own packaging, the membrane 132 is soaked in the lubricant 136. In that regard, the membrane 132 is a separate component that can be later attached to the disposable probe 101 (e.g., to the concave surface 130). The membrane 132 can be attached to the disposable probe 101 (e.g., to an edge of the concave surface 130) via adhesives, hoop-and-look fasteners, latches, hooks, and so on.

Because the disposable probe 101 is disposable, the membrane 132 with the gel 134 therein can be utilized, since the disposable probe 101 itself is needed for a relatively short period of time (e.g., one use). Accordingly, use of the membrane 132 frees an operator from having to use and apply gel on a subject to improve ultrasound transmission.

In other arrangements, instead of the concave surface 130, the disposable probe 101 has a convex surface (e.g., a convex lens) protruding from the top end of the disposable probe 101. Given that the surface is convex, gel may not be needed between the convex surface and the scanning surface due to the lack of air gaps allowed between the protruding surface of the convex surface and the scanning surface (e.g., direct contact is made between the two surfaces).

In some arrangements, because a structural interface can be formed between the disposable probe 101 and the base 102, there are opportunities to insert additional components between the disposable probe 101 and the base 102, which is not afforded by permanent probe systems. The additional components allow additional functionalities of the probe system 100. In some arrangements, a force sensor or pressure sensor can be provided between the base 102 and the disposable probe 101 to allow force or pressure readings of force exerted on the surface of the disposable probe 101 (e.g., where contact is made with a scanning surface). For example, the force sensor can be coupled to the base 102 or to the disposable probe 101 before the base 102 is attached to the disposable probe 101 in the manner described. In other arrangements, the ultrasound system includes the force sensor already at the base 102 as part of the base 102 (e.g., at the surface of the base 102 where the base 102 is configured to receive the disposable probe 101).

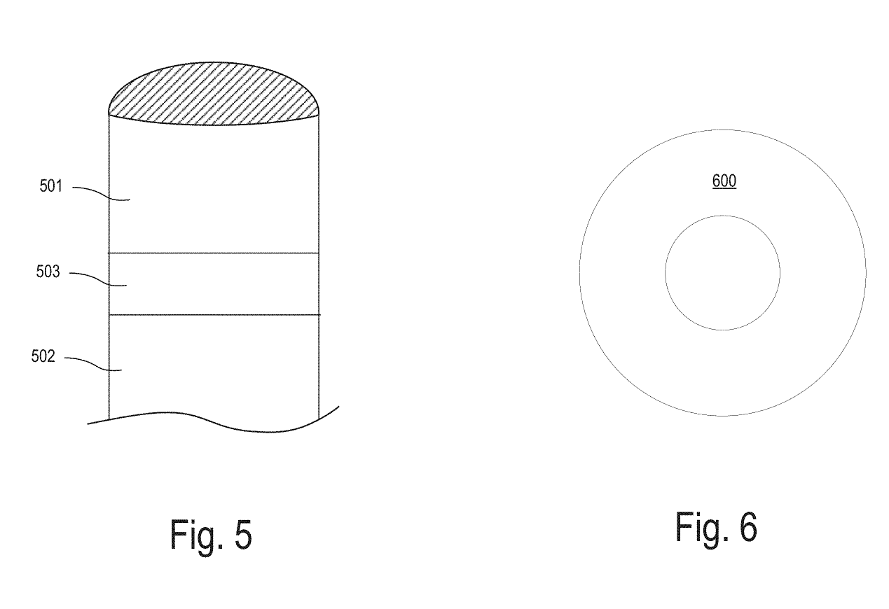

In one example, the force sensor is a load cell positioned between the base 102 and the disposable probe 101 for detecting an amount of reactionary force that the scanning surface exerts against the disposable probe 101 as the disposable probe 101 exerts an opposite force against the scanning surface. In an example in which the disposable probe 101 (when attached to the base 102 and moved by the robotic system as described herein) utilizes physical contact against a subject for operation, the load cell can detect an amount of force exerted by the scanning surface against the disposable probe 101 (e.g., against the membrane 132). For instance, FIG. 5 shows an example implementation of a load cell 503 of a probe system 500 according to various arrangements. An example of the probe system 500 is the probe system 100, where details of the probe system 100 are omitted. The probe system 100 includes a disposable probe 501 (such as but not limited to, the disposable probe 101) and a base 502 (such as but not limited to, the base 202). When the disposable probe 501 and the base 502 are removably coupled, the load cell 503 is between the disposable probe 501 and the base 502 to transmit force exerted to the disposable probe 501 to the base 502. In other arrangements, the load cell 503 can be behind the base 502 such that base 502 is interposed between the disposable probe 501 and load cell 503. For example, the load cell 503 can be coupled to the base 502 or to the disposable probe 501 before the base 502 is attached to the disposable probe 501. The load cell 503 can be configured to measure the force exerted by the scanning surface against the disposable probe 501 in one or more of a first axis along or parallel to a central axis of a body (e.g., cylindrical shape) of the disposable probe 501 and, in some arrangement, additional Cartesian and rotational axes perpendicular to the first axis. Measuring the force in the first axis corresponds to measuring a force normal to the scanning surface (e.g., the skin of the subject). Measuring the force in the additional axes corresponds to measuring forces exerted to the disposable probe 501 by objects (e.g., hair, ear, the scanning surface as the probe is dragged there along, and so on) at the scanning surface, or by the scanning surface itself.

In some examples, the disposable probe 501 includes a force transmitter configured to transmit force exerted on the disposable probe 501 to a load cell disposed on the base 502. FIG. 6 shows an example implementation of a force transmitter 600 according to various arrangements. The force transmitter (e.g., the force transmitter 600) can be mechanical and include a piece of rigid material (e.g., metal, metal alloy, a rigid plastic, and so on) having a shape (e.g., a ring, a circle, and so on) configured to transmit the force in one or more axes. As shown, the force transmitter 600 (shown in a front view in FIG. 6) is a ring or toroid. The force transmitter is rigidly fixed to a housing of the disposable probe 501 such that the force transmitter moves with the housing. In the examples in which the force transmitter (e.g., the force transmitter 600) is a ring or a circle, the center of the ring or the circle is on a central axis of a cylindrical shape of the disposable probe 501. The force transmitter is configured to be friction fitted to the load cell when the disposable probe 501 is attached to the base 502. At least a portion of the load cell 503 has a shape corresponding to the force transmitter 600. When the disposable probe 501 is attached to the base 502, the force exerted on the housing, the membrane 132 (omitted in FIG. 5), and the surface 130 (omitted in FIG. 5) is transmitted to the force transmitter 600, and the force transmitter 600 transmits the substantially same or same amount of force to the load cell on the base 502. In the examples in which the force transmitter (e.g., the force transmitter 600) is a ring or a circle, the force exerted along the central axis of the body (e.g., cylindrical shape) of the disposable probe 501 is transmitted by movement of the ring or the circle along the central axis. In additional arrangements, the force exerted in other axes different from the central axis is transmitted by movement of the ring or the circle in those axes.

The data corresponding to the detected force can be sent by the load cell to a controller for processing. In some arrangements, the controller is located at the system (e.g., ultrasound system) that includes or is attached to the base 102. Based on the detected force, the controller can regulate the force that the disposable probe 101 exerts against the subject so that the disposable probe 101 is not causing pain or discomfort to the subject or to ensure that the disposable probe 101 is pressed against the subject using an adequate amount of force so that the disposable probe 101 can adequately perform the functionalities described herein (e.g., transmit and/or receive suitably strong ultrasound signals).

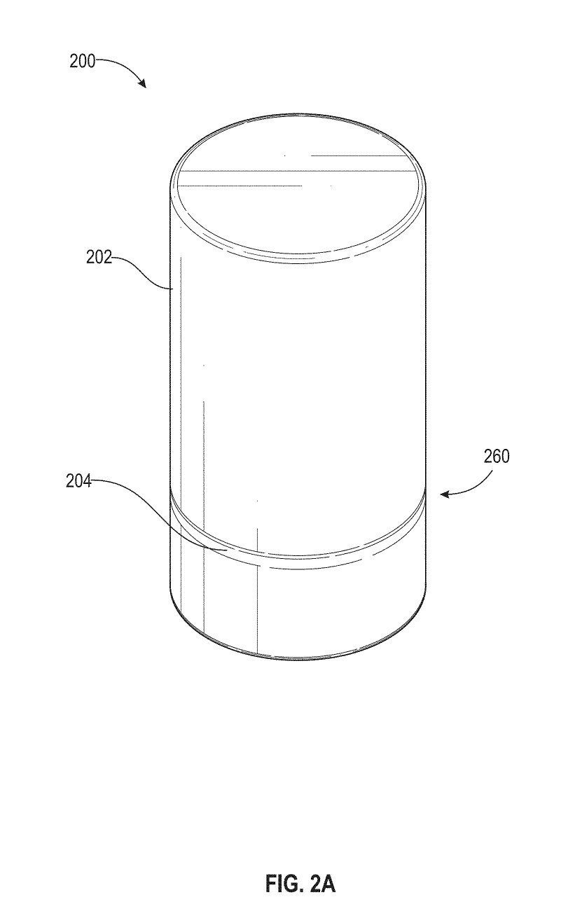

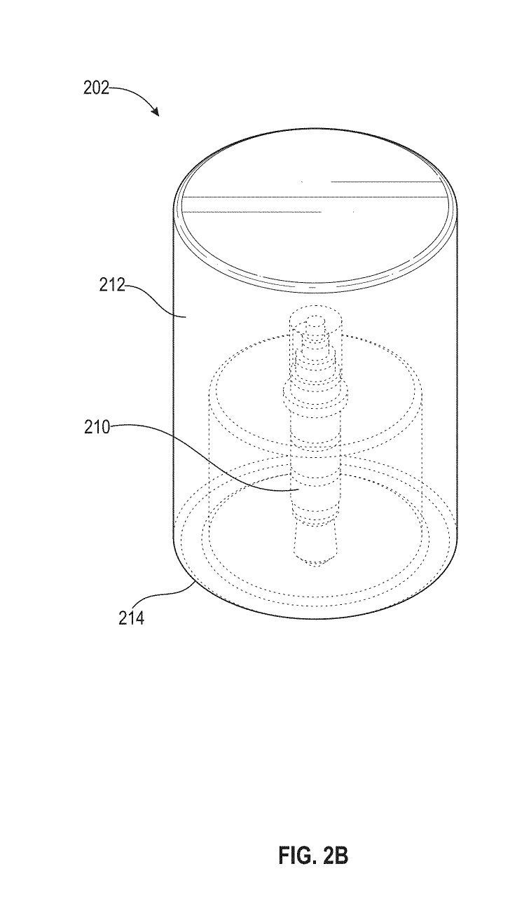

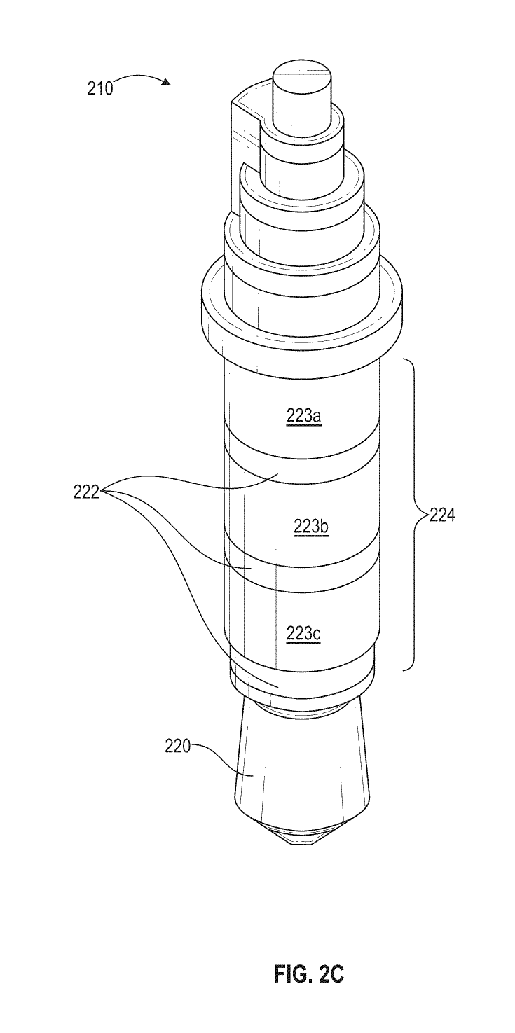

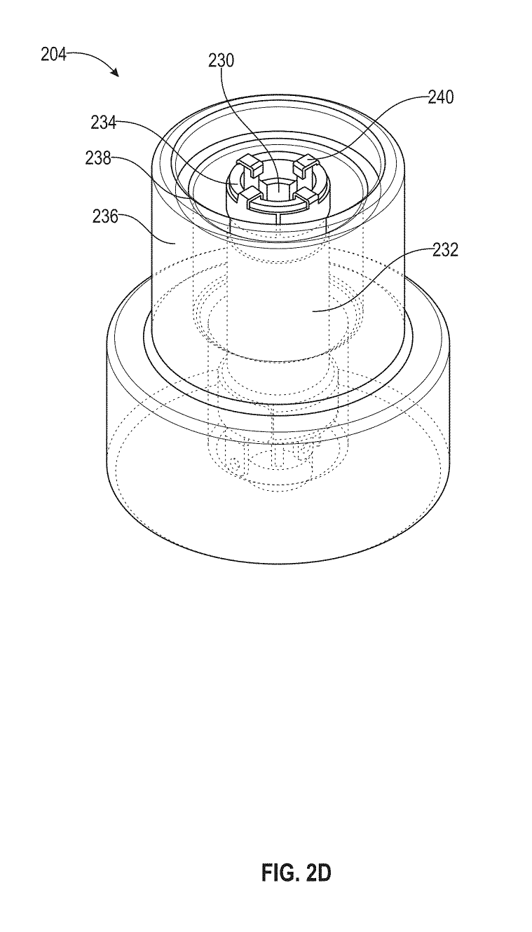

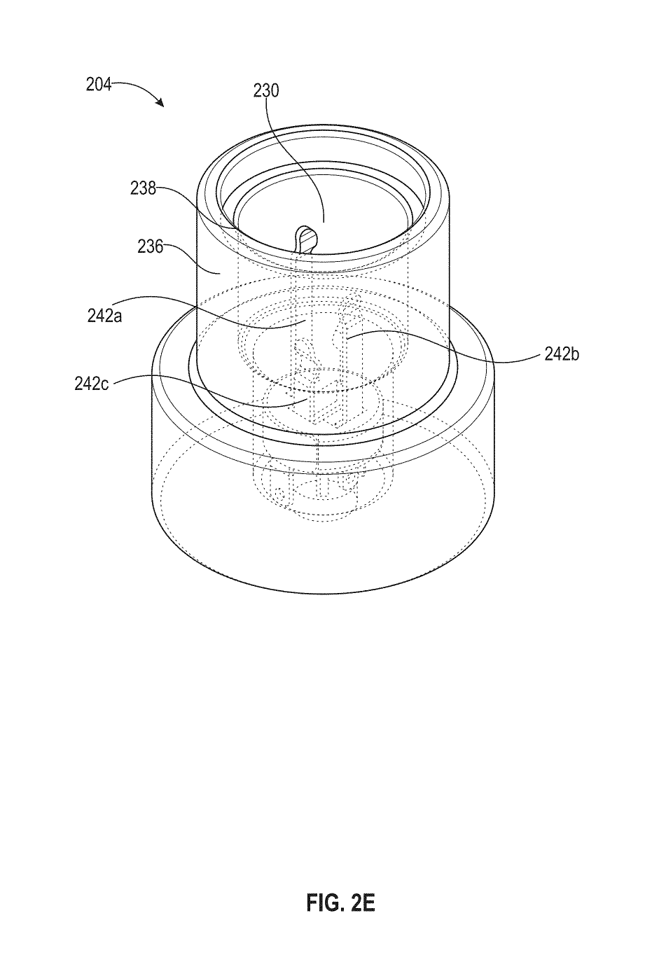

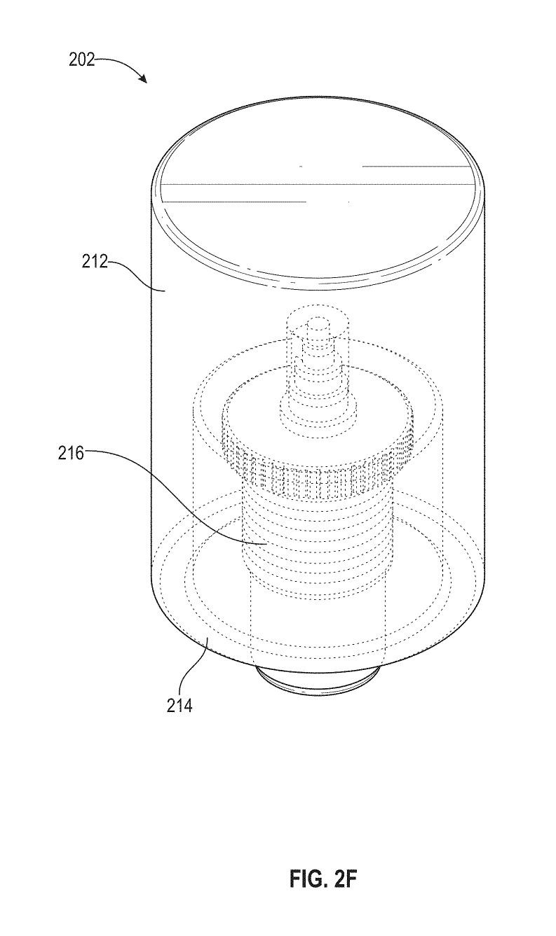

FIGS. 2A-2F show an example of a probe system 200 according to various arrangements. Referring to FIGS. 2A-2F, the probe system 200 includes a disposable probe (or a disposable component of the probe) 202 and a base 204. In some arrangements, the description with respect to the disposable probe 101 and the base 102 above can be applied to the disposable probe 202 and the base 204, respectively. In some arrangements, the disposable probe 202 is configured to be one or more of a camera, an ultrasound instrument, a thermal instrument, a near-infrared instrument, an optical camera instrument, a lighting instrument, a spectrometer instrument, a microphone instrument, an electroencephalography instrument, an electromagnetic instrument, and so on. While an ultrasound instrument is used as an example of the disposable probe 202, the disposable probe 202 can be another device listed herein. The disposable probe 202 may include a single sensor/instrument or an array (e.g., a linear array, a circular array, and so on) of sensors/instruments. disposable probe 202 may include electronic components, a concave surface, a convex surface, and so on similar to described with respect to the disposable probe 101.

In some arrangements, the base 204 is part of a manually-operated system for positioning the disposable probe 202. In other arrangements, the base 204 is part of a robotic system (not shown) for positioning the disposable probe 202 similar to described with respect to the base 102.

In some arrangements, the disposable probe 202 is configured to be releasably attached to the base 204. That is, the disposable probe 202 can be removably attached to the base 204 such that the disposable probe 202 can be attached to and detached from the base 204 by an operator without using tools.

In some arrangements, the disposable probe 202 includes a jack 210 (e.g., a male portion of a jack, a male connector, a male thread, and so on), which is a connection interface of the disposable probe 202. In some arrangements, the jack 210 extends from a top end of the disposable probe 202 toward a bottom end of the disposable probe 202, where the bottom end of the disposable probe 202 is closer to a top end of the base 204 than the top end of the disposable probe 202 when the disposable probe 202 is attached to the base 204. In some arrangements, the jack 210 is located at a central portion of the disposable probe 202 (e.g., along a central axis of a cylindrical shape of the disposable probe 202) as shown. In other arrangements, the jack 210 is located at a side portion of the disposable probe 202, where the side portion is offset from a center (e.g., the central axis) of the disposable probe 202. In some arrangements, a first end of the jack 210 is attached (e.g., permanently or releasably attached) at the top end of the disposable probe 202 and a second end of the jack 210 opposite the first end is free when the disposable probe 202 is not connected to the base 204.

In some examples, the jack 210 includes a tip 220, one or more rings (e.g., rings 222), and a sleeve 224. The tip 220 is on the second end of the jack 210. In some arrangements, the rings 222 are located at various locations along the sleeve 224. The rings 222 isolate one or more sections 223a-223c of the sleeve 224 and the tip 220 from each other, allowing different types of signals (e.g., data signals, power, control signals, ground, and so on) to be transmitted or received using the jack 210. The data signals include data collected by the disposable probe 202. The control signals include signals sent by the system attached to the base 204 to the disposable probe 202 configured to control data collection (e.g., energy generation and collection) functionalities of the disposable probe 202. In some arrangements, the sections 223a-223c and the rings 222 encompass the entire circumference of the jack 210. In other arrangements, the sections 223a-223c and the rings 222 encompass less than the entire circumference of the jack 210. In some arrangements, each of the sections 223a-223c transmits or receives a different type of signals (e.g., a different one of the data signals, the power, the control signals, the ground, and so on). In other arrangements, two or more of the sections 223a-223c transmit a same signal. While the three sections 223a-223c are shown, any number of sections can be provided on the sleeve 224.

In other arrangements, any suitable shape and configuration of the jack 210 can be implemented for adequately transmitting and/or receiving signals therethrough. For example, instead of the one or more sections 223a-223c being stacked along the length of the jack 210, the jack 210 can be flat and rectangular-shaped and the sections 223a-223c can be aligned in a row along a width of the jack 210 for receiving or transmitting their respective signals.

In some arrangements, the disposable probe 202 includes an outer wall 212 that surrounds and extends past the jack 210. While the outer wall 212 is shown to have a cylindrical shape, the outer wall 212 may have any other suitable shape such as but not limited to, a cuboid shape, an oval shape, and so on. The disposable probe 202 can also include a bottom surface 214 which connects the outer wall 212. The bottom surface 214 or at least a portion thereof is configured to face and/or contact a surface of the base 204.

In some arrangements, the base 204 includes a connection interface having a cavity 230 surrounded by an inner shell 232, an upper ring 234 connected to the inner shell 232, an outer shell 236 surrounding the inner shell 232, and a top surface 238 connected to the outer shell 236. The top surface 238 is configured to face and/or contact the bottom surface 214 in some examples. The upper ring 234 includes a plurality of teeth 240 extending inwards in some examples. The base 204 (the connection interface thereof) can include a plurality of electrical spring contacts 242a-242c within the cavity 230. The electrical spring contacts 242a-242c can be enclosed by the inner shell 232 (e.g., as shown in FIG. 2D). The disposable probe 202 can attach to the base 204 by inserting the jack 210 (e.g., the second end of the jack 210) into the cavity 230. Upon inserting the jack 210 into the cavity 230, the jack 210 can be securely held by the plurality of teeth 240 of the upper ring 234 and the plurality of electrical spring contacts 242a-242c. Upon insertion, the plurality of electrical spring contacts 242a-242c can electrically couple to the sections 223a-223c of the sleeve 224 and the tip 220.

In some arrangements, each of the sections 223a-223c is configured to couple to a corresponding one of the electrical spring contracts 242a-242c. Accordingly, in some arrangements, each of the electrical spring contacts 242a-242c extends at different heights from the bottom of the base 204 so as to contact a different one of the sections 223a-223c of the sleeve 224. For example, the electrical spring contact 242a is configured to contact or otherwise electrically couple to the section 223a. The electrical spring contact 242b is configured to contact or otherwise electrically couple to the section 223b. The electrical spring contact 242c is configured to contact or otherwise electrically couple to the section 223c. This allows three different types of signals (e.g., three of the data signals, the power, the control signals, the ground, and so on) to be communicated between the base 204 and the disposable probe 202.

In other arrangements, two or more of the electrical spring contacts 242a-242c are configured to contact a same one of the sections 223a-223c. Although three rings 222 and three sections 223a-223c are shown, more or less rings and sections of the sleeve 224 can be implemented. In some arrangements, the number of the rings and the number of sections of the sleeve 224 are the same. In other arrangements, the number of the rings and the number of sections of the sleeve 224 are different.

In some arrangements, the tip 220 of the jack 210 is configured to contact another electrical contact at the bottom of the cavity 230 such that the tip 220 is in electrical communication with the bottom of the base 204. The bottom of the base 204 may include an electrical contact. Accordingly, the tip 220 and the bottom of the base 204 can form an additional electrical connection when the jack 210 is inserted into the cavity 230, allowing an additional signal type (e.g., an additional one of the data signals, the power, the control signals, the ground, and so on) to be communicated between the system including the base 204 and the disposable probe 202.

In some examples, the base 204 can include a plane such as a printed circuit board (PCB) that the base 204 is mounted onto (not shown). The electrical spring contacts 242a-242c may be soldered onto the plane. The sections 223a-223c and the tip 220 of the jack 210 and the electrical spring contacts 242a-242c can be made of conductive material such as but not limited to, a metal (e.g., copper), a metal alloy, and so on. The rings 222 of the jack 210 may be made of an insulating material such as but not limited to, a plastic, rubber, and so on.

In some arrangements, additional mechanisms improve the strength of the mechanical connection. In some arrangements, the disposable probe 202 can include a male threaded sleeve 216 surrounding the jack 210. The male threaded sleeve 216 is enclosed by the outer wall 212. The base 204 can have a corresponding female threaded inner surface (not shown) of the outer shell 236. When the disposable probe 202 is brought into contact with the base 204, the disposable probe 202 can be rotated so the male threaded sleeve 216 can be screwed into the female threaded inner surface of the outer shell 236. In other arrangements, the base 204 includes magnets while the disposable probe 202 includes corresponding metal attraction contacts for aligning with the magnets (or vice versa), for example, at locations where the base 204 and the disposable probe 202 interface (e.g., contact each other).

In some arrangements, the disposable probe 202 includes one or more sensors including but not limited to, one or more pressure sensors, one or more temperature sensors, or one or more of any other sensors. The one or more sensors may be located on the bottom surface 214 of the disposable probe 202. Upon attaching the disposable probe 202 to the base 204, top surface 238 of the base 204 applies pressure to the one or more sensors. The one or more sensors may be located on the concave surface of the disposable probe 202 (similar to shown in FIG. 1) to sense the pressure of the membrane including the gel that is attached to the disposable probe 220. In other arrangements, the one or more sensors are located along an edge or a side surface of the head of the disposable probe 202 so that the sensors do not interfere with the signal transmitted and received at the end surface of the disposable probe 202 (e.g., the concave surface). In some arrangements, the one or more sensors convert a non-electrical signal, such as pressure, sound, and the like, into an electrical signal, such as voltage, current, resistance, and the like. The one or more sensors may be implemented by a strain gauge, a capacitive sensor, an electrode, and the like. The capacitive sensor may include a piezoelectric transducer and a flexible membrane whose capacitance changes as a function of pressure applied to the flexible membrane.

In some arrangements, the disposable probe 202 includes one or more light emitting diodes (LEDs). Each of the one or more LEDs may be electrically coupled to one of the one or more sensors. If one of the one or more electrical signals generated by the one or more sensors is above a pre-determined threshold, a corresponding one of the one or more LEDs can emit a light. The one or more LEDs may be located on an outer surface of the outer wall 212 of the disposable probe 202. In alternative arrangements, the base 204 includes the one or more LEDs. The electrical signals of the one or more sensors can be electrically coupled to the one or more LEDs through the one or more sections of the sleeve 224 of the jack 210 and the corresponding plurality of electrical spring contacts 242. The LEDs may be located on an outer surface of the outer shell 236 of the base 204. The LEDs may be located on the plane of the base 204.

In some arrangements, the disposable probe 202 includes one or more liquid-crystal displays (LCDs) or other suitable display device. Each of the one or more LCDs can display one or more values corresponding to some or all of the one or more non-electrical signals of the one or more sensors. For example, the LCDs can display a pressure reading. In other arrangements, the base 204 includes the one or more LCDs.

In some arrangements, the disposable probe 202 is of a first type. The type of a disposable probe may be defined by frequency of operation (e.g., 2, megahertz, 10 megahertz, 50 megahertz, and so on), sensor features, type of energy waves emitted (e.g., ultrasound, infrared, and so on), type of modulation or multiplexing scheme modulating or multiplexing a carrier of the energy wave (e.g. amplitude modulation, frequency modulation, phase modulation, amplitude-shift keying, frequency-shift keying, phase-shift keying, quadrature amplitude modulation, quadrature phase-shift keying, time division multiplexing, frequency division multiplexing, orthogonal frequency division multiplexing, code division multiple access, and so on), and the like. The disposable probe 202 may be a member of a plurality of different types of disposable probes.

The base 204 may be mechanically keyed to the first type of disposable probes, thus keyed to the disposable probe 202, to allow proper installation of the disposable probe 202. This prevents an operator installing a disposable probe of another type to the base 204. In some arrangements, the outer shell 236 of the base 204 and the outer wall 212 of the disposable probe 202 (the first type) are shaped such that the base 204 only properly attaches to the first type of disposable probes (including the disposable probe 202). In other arrangements, the first type of disposable probes (e.g., the disposable probe 202) has a corresponding pre-defined number of rings (e.g., the rings 222). The base 204 may only properly attach if the pre-defined number of the rings 222 of the disposable probe 202 has a pre-defined numerical relationship with a number of the electrical spring contacts 242 in the base 204. For example, the number of the electrical spring contacts 242 can be one greater than the number of rings 222 for a proper attachment. In other arrangements, other methods of mechanical keying can be implemented, such as a predefined pattern of notches at the disposable probe 202 matches corresponding features (e.g., protrusions or recesses) at the base 204. In other arrangements, mechanical keying is accomplished by shapes of the extending portion of the disposable probe 202 (e.g. the outer wall 212) that slides into a corresponding slot at the base 204 (e.g., both the extending portion and the slot are square shaped, triangular, circular, and so on) or around the outer shell 236 (e.g. both the extending portion and the outer shell 236 are square shaped, triangular, circular, and so on). In some arrangements, the first type of disposable probes is mechanically keyed differently from a second type of disposable probes.

In yet other arrangements, the bottom surface 214 of the disposable probe 202 includes protruding pins or leads or pogo pins (e.g., similar to those shown in FIG. 1) that are received by corresponding recessed contacts (e.g., similar to those shown in FIG. 1) at the base 204. In some arrangements, the connections between the protruding pins and the recessed contacts close the electrical connections between the disposable probe 202 and the base 204 to allow transmission of information between the disposable probe 202 and the base 204. In some arrangements, the location of the protruding pins is specific to the first type of disposable probes. Corresponding recessed contacts on the base 204 properly attach to a disposable probe (e.g., the disposable probe 202) of the first type. In some arrangements, the base 204 includes an irregular, maze-like plurality of recessed contacts such that disposable probe 202 must be rotated and attached in a pre-defined way in order for the protruding pins of the disposable probe 202 to make contact with the recessed contacts of the base 204. In some arrangements, if the protruding pins properly make contact with the recessed contacts, a corresponding event occurs notifying a user that proper alignment has occurred (e.g., an LED emits a first colored light, a sound is emitted, a tactile response, and so on). In some arrangements, if the pogo pins do not make proper contact with the recessed contacts, a corresponding event occurs to notify a user that improper alignment has occurred (e.g., an LED emits a second colored light, a sound is emitted, a tactile response, and so on).

In other arrangements, the base 204 may be keyed to the disposable probe 202 via identifiers stored in memory. In some arrangements, the disposable probe 202 includes a memory device. The memory device may be located at the top of the disposable probe 202 and may be electrically coupled to the first end of the jack 210. The base 204 or the system that is attached to or includes the base 202 may include a processor. The processor can be located at the bottom of the base 204 and may be electrically coupled to a first end of one of the electrical spring contacts 242. Upon attaching the disposable probe 202 to the base 204, the electrical contact 242 can make contact with one of the sections of the sleeve 224 of the jack 210, and the processor can read from and write to addresses in the memory device. The processor of the base 204 can identify the disposable probe 202 as a disposable probe of the first type by reading information from the memory device. For example, the disposable probe 202 may have a unique identifier stored on the memory device. In another example, the memory device may have stored thereon the frequency of operation of the disposable probe 202. In some arrangements, if the base 204 identifies that the disposable probe 202 is compatible with the base 204, an event occurs notifying a user that a compatible disposable probe 202 has been connected (e.g., an LED emits a first colored light, a sound is emitted, a tactile response, and so on). In some arrangements, if the base 204 determines that the disposable probe 202 is incompatible with the base 204, a corresponding LED emits a second colored light. In other arrangements, the processor is located at the disposable probe 202 and the memory is located at the base 204 or the memory and the processor are both located at either the disposable probe 202 or the base 204.

In some examples, a connection location 260 corresponds to a location where a surface of the disposable probe 202 and a surface of the base 204 (facing the surface of the disposable probe 202) contact or face each other when the connection interface of the disposable probe 202 and the connection interface of the base 204 are removably attached. The connection location 260 represent locations of the connection interface of the disposable probe 202 and the connection interface of the base 204. In some examples, the connection location 260 is configured to be within a predetermined distance (e.g., 3 inches, 4 inches, 6 inches, 12 inches, and so on) from a scanning surface (e.g., a skin of a subject) during operations of the disposable probe 202 (e.g., when the connection interface of the disposable probe 202 and the connection interface of the base 204 are removably attached).

When the disposable probe 202 is removably attached to the base 204, the length of the probe system 200 (including the length of the disposable probe 202 and the length of the base 204) is less than a predetermined length (e.g., 3 inches, 5 inches, 7 inches, 8 inches, 10 inches, 12 inches, and so on). The location of the connection interfaces of the disposable probe 202 and the base 204 is within a predetermined distance (e.g., 0.5 inch, 1 inch, 1.5 inches, 2 inches, 3 inches, and so on) from an end of the disposable probe 202 opposite to the end on which the connection interface of the disposable probe 202 is located. The location of the connection interfaces of the disposable probe 202 and the base 204 is within a predetermined distance (e.g., 1 inch, 1.5 inches, 2 inches, 3 inches, 4 inches, and so on) from an end of the base 204 that is opposite to the end of the base 204 on which the connection interface of the base 204 is located. In some examples, the location of the connection interfaces of the disposable probe 202 and the base 204 is within a predetermined distance (e.g., 0.5 inch, 1 inch, 1.5 inches, 2 inches, 3 inches, and so on) from both the end of the disposable probe 202 (that is opposite to the end on which the connection interface of the disposable probe 202 is located) and the end of the base 204 (that is opposite to the end of the base 204 on which the connection interface of the base 204 is located).

That is, the location of the connection interfaces is equidistant from the end of the disposable probe 202 (that is opposite to the end on which the connection interface of the disposable probe 202 is located) and the end of the base 204 (that is opposite to the end of the base 204 on which the connection interface of the base 204 is located). In some examples, the location of the connection interfaces is closer to the end of the disposable probe 202 (that is opposite to the end on which the connection interface of the disposable probe 202 is located) than to the end of the base 204 (that is opposite to the end of the base 204 on which the connection interface of the base 204 is located).

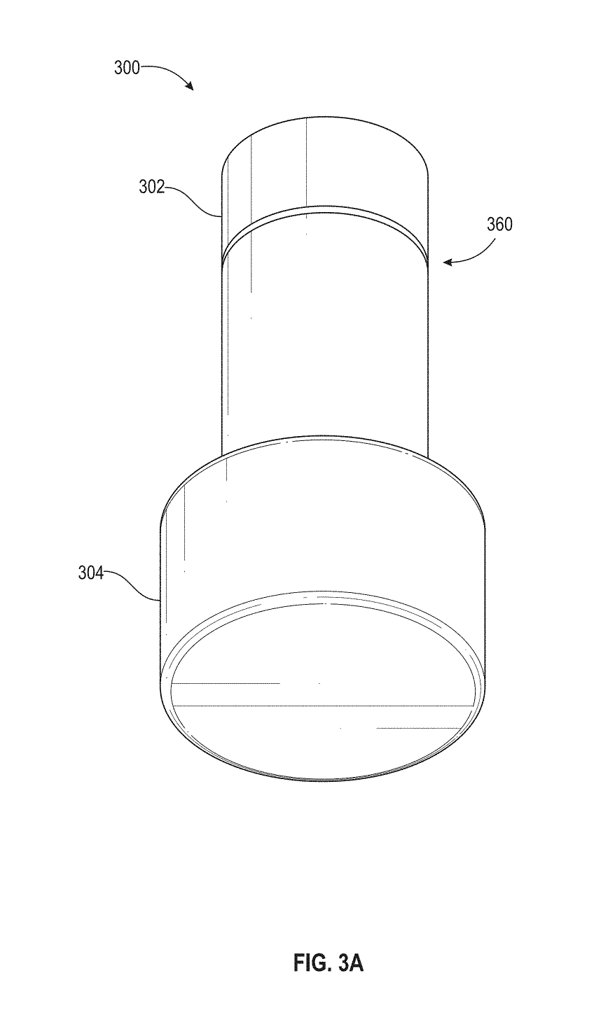

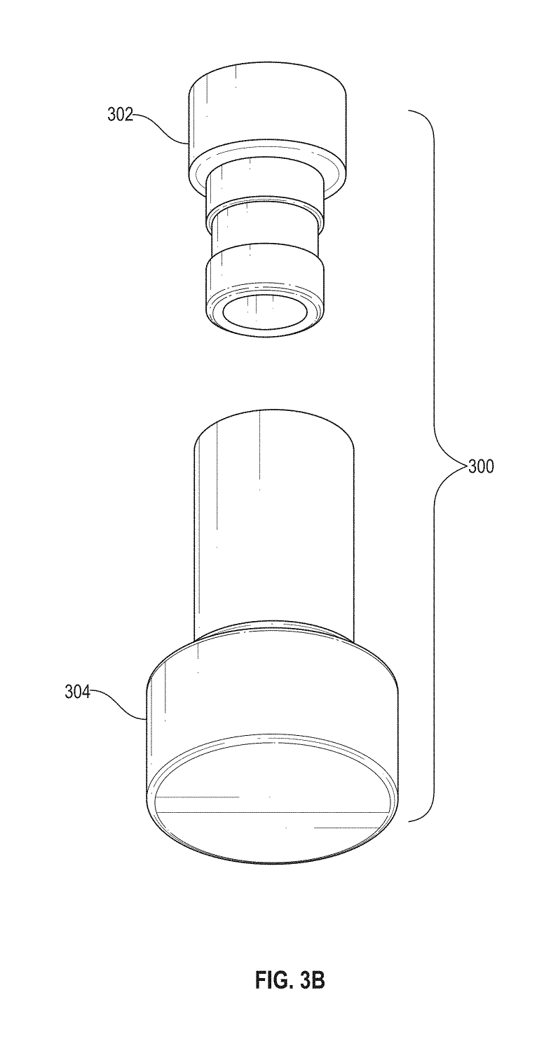

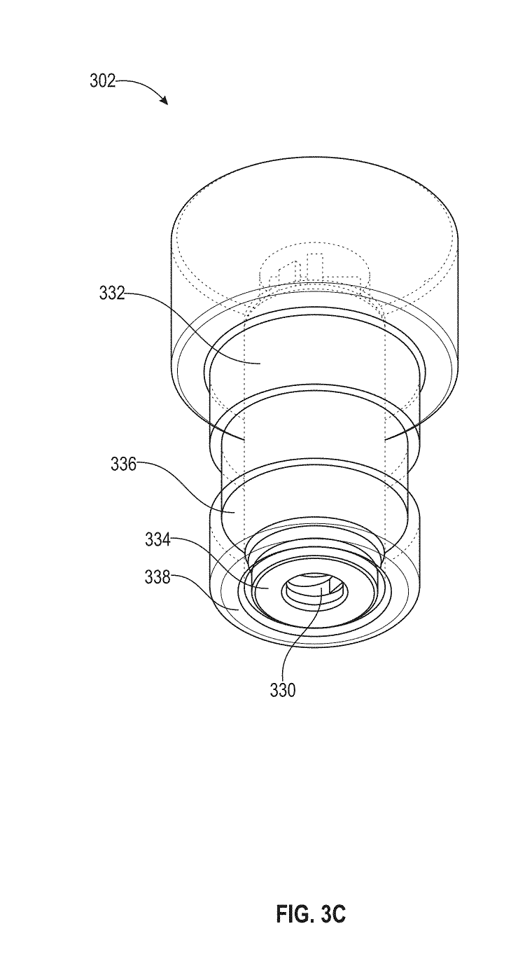

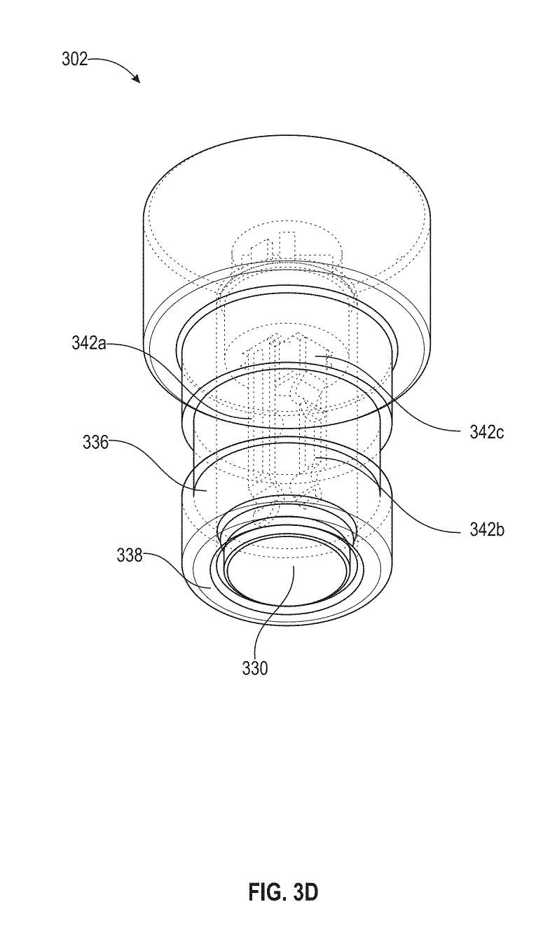

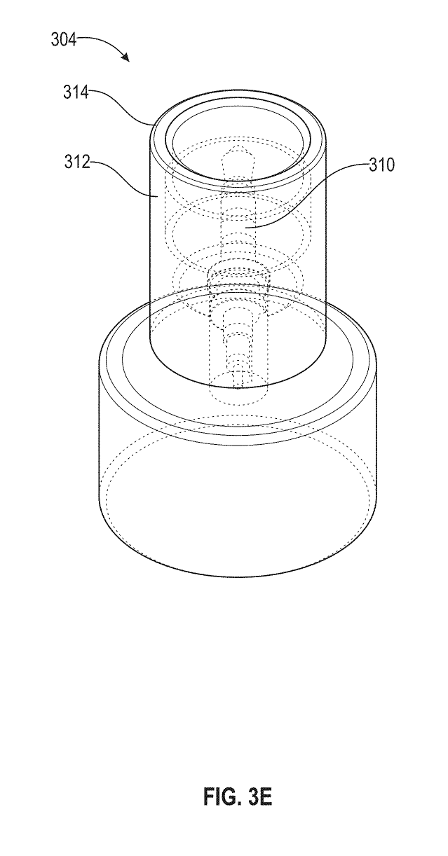

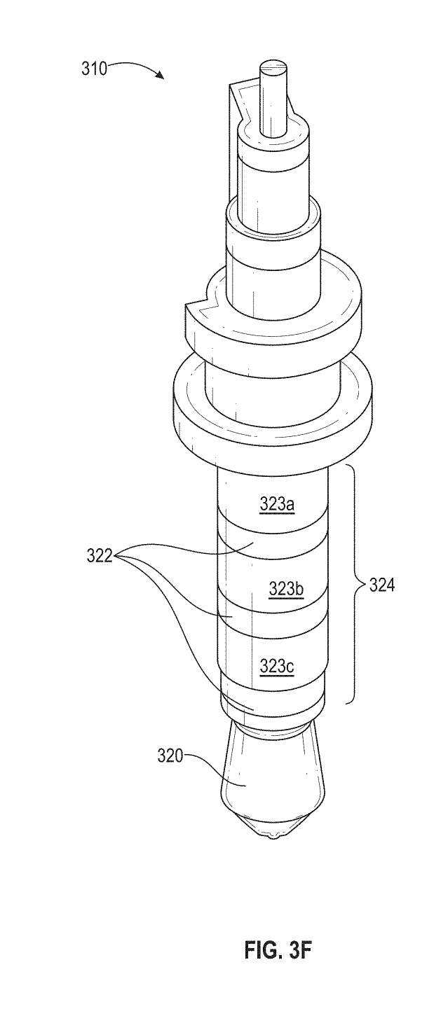

FIGS. 3A-3F show an example of a probe system 300 according to various arrangements. Referring to FIGS. 3A-3F, the probe system 300 includes a disposable probe (or a disposable component of the probe) 302 and a base 304. In some arrangements, the disposable probe 302 is configured to be one or more of a camera, an ultrasound instrument, a thermal instrument, a near-infrared instrument, an optical camera instrument, a lighting instrument, a spectrometer instrument, a microphone instrument, an electroencephalography instrument, an electromagnetic instrument, and so on. While an ultrasound instrument is used as an example of the disposable probe 302, the disposable probe 302 can be another device listed herein. The disposable probe 302 may include a single sensor/instrument or an array (e.g., a linear array, a circular array, and so on) of sensors/instruments. The disposable probe 302 may include electronic components, a concave surface, a convex surface, and so on similar to described with respect to the disposable probe 101.

In some arrangements, the base 304 is part of a manually-operated system for positioning the disposable probe 302. In other arrangements, the base 304 is part of a robotic system (not shown) for positioning the disposable probe 302 similar to described with respect to the base 102.

In some arrangements, the disposable probe 302 is configured to be releasably attached to the base 304. That is, the disposable probe 302 can be removably attached to the base 304 such that the disposable probe 302 can be attached to and detached from the base 304 by an operator without using tools.

In some arrangements, corresponding elements described above with respect to the probe system 200 (e.g., FIGS. 2A-2F) are applicable to the elements described below with respect to FIG. 3. FIG. 3B illustrates an exploded view of the disposable probe 302 and the base 304. The disposable probe 302 can include a connection interface having a cavity 330 surrounded by an inner shell 332, a lower ring 334 connected to the inner shell 332, an outer shell 336 surrounding the inner shell 332, and a bottom surface 338 connected to the outer shell 336. The disposable probe 302 (the connection interface thereof) can include a plurality of electrical spring contacts 342a-342c surrounding the cavity 330. The electrical spring contacts 342a-342c can be enclosed by the inner shell 332.

In some arrangements, the base 304 includes connection interface having a jack 310. In some examples, the jack 310 includes a tip 320, one or more rings (e.g., rings 322), and a sleeve 324. The rings 322 are located at various points along the sleeve 324, and the rings 322 isolate one or more sections 323a-323c of the sleeve 324 and the tip 320 from each other, allowing different types of signals (e.g., data signals, power, control signals, ground, and so on) to be transmitted using the jack 310. The base 304 can include an outer wall 312 that surrounds the jack 310. The base 304 can also include a top surface 314 which connects to the outer wall 312.

In some arrangements, each of the sections 323a-323c is configured to couple to a corresponding one of the electrical spring contracts 342a-342c. Accordingly, in some arrangements, each of the electrical spring contacts 342a-342c extends at different heights from the top of the disposable probe 302 so as to contact a different one of the sections 323a-323c of the sleeve 324. For example, the electrical spring contact 342a is configured to contact or otherwise electrically couple to the section 323a. The electrical spring contact 342b is configured to contact or otherwise electrically couple to the section 323b. The electrical spring contact 342c is configured to contact or otherwise electrically couple to the section 323c. This allows three different types of signals (e.g., three of the data signals, the power, the control signals, the ground, and so on) to be communicated between the base 304 and the disposable probe 302.

In other arrangements, two or more of the electrical spring contacts 342a-342c are configured to contact a same one of the sections 323a-323c. Although three rings 322 and three sections 323a-323c are shown, more or less rings and sections of the sleeve 324 can be implemented. In some arrangements, the number of the rings and the number of sections of the sleeve 324 are the same. In other arrangements, the number of the rings and the number of sections of the sleeve 324 are different. The interactions, coupling, and characteristics of the electrical spring contacts 342a-342c and the sections 323a-323c are similar to described with respect to the electrical spring contacts 242a-242c and the sections 223a-223c.

In some arrangements, the disposable probes 202 and 302 each includes the coupling layer 120 of the disposable probe 101 as shown in FIG. 1, the first metallic layer 122 of the disposable probe 101 as shown in FIG. 1, the PZT layer 124 of the disposable probe 101 as shown in FIG. 1, the second metallic layer 126 of the disposable probe 101 as shown in FIG. 1, the concave surface 130 of the disposable probe 101 as shown in FIG. 1, the membrane 132 including the gel 134 therein of the disposable probe 101 as shown in FIG. 1, or a combination thereof.

In particular arrangements where the disposable probe 302 includes the cavity 330 and the base 304 includes the jack 310, the disposable probe 302 may contain a membrane covering the cavity 330. Accordingly, upon attaching the disposable probe 302 to the base 304, the jack 310 pierces through the membrane. The membrane pushes any excess gel or other fluid previously used with respect to the base 304 downward and away from the electrical spring contacts 342a-342c of the disposable probe 302 and downwards along the jack 310 such that the gel or other fluid is removed so that it does not interfere with electrical communication between the disposable probe 302 and the base 304. In some arrangements, the base 304 can include weep holes at a bottom surface of the cavity 330. The gel can be siphoned through the weep holes. The membrane may be made of silicone material. In addition, by including the piercable membrane, a user knows that a particular disposable probe 302 was used based on whether the membrane was pierced or not. This membrane can also be used with disposable probes 101 and 202.

In some arrangements, the disposable probe 302 includes one or more sensors (including but not limited to, one or more pressure sensors, one or more temperature sensors, or one or more of any other sensors) similar to described with respect to the disposable probe 202. In some arrangements, the disposable probe 302 includes one or more LEDs similar to described with respect to the disposable probe 202. In some arrangements, the disposable probe 302 includes one or more LCDs or other suitable display device similar to described with respect to the disposable probe 202. In some arrangements, the base 304 may be mechanically keyed to a second type of disposable probes, where the disposable probe 202 is of the second type, similar to described with respect to the disposable probe 202 and the base 204. In some arrangements, the disposable probe 302 includes protruding pins or leads or pogo pins (e.g., similar to those shown in FIG. 1 or described with respect to the disposable probe 202) that are received by corresponding recessed contacts (e.g., similar to those shown in FIG. 1 and described with respect to the base 204) at the base 304. In some arrangements, the base 304 may be keyed to the disposable probe 302 via identifiers stored in memory similar to described with respect to the disposable probe 202 and the base 204.

In some examples, a connection location 360 corresponds to a location where a surface of the disposable probe 302 and a surface of the base 304 (facing the surface of the disposable probe 302) contact or face each other when the connection interface of the disposable probe 302 and the connection interface of the base 304 are removably attached. The connection location 360 represent locations of the connection interface of the disposable probe 302 and the connection interface of the base 304. In some examples, the connection location 360 is configured to be within a predetermined distance (e.g., 3 inches, 4 inches, 6 inches, 12 inches, and so on) from a scanning surface (e.g., a skin of a subject) during operations of the disposable probe 302 (e.g., when the connection interface of the disposable probe 302 and the connection interface of the base 304 are removably attached).

When the disposable probe 302 is removably attached to the base 304, the length of the probe system 300 (including the length of the disposable probe 302 and the length of the base 304) is less than a predetermined length (e.g., 3 inches, 5 inches, 7 inches, 8 inches, 10 inches, 12 inches, and so on). The location of the connection interfaces of the disposable probe 302 and the base 304 is within a predetermined distance (e.g., 0.5 inch, 1 inch, 1.5 inches, 2 inches, 3 inches, and so on) from an end of the disposable probe 302 opposite to the end on which the connection interface of the disposable probe 302 is located. The location of the connection interfaces of the disposable probe 302 and the base 304 is within a predetermined distance (e.g., 1 inch, 1.5 inches, 2 inches, 3 inches, 4 inches, and so on) from an end of the base 304 that is opposite to the end of the base 304 on which the connection interface of the base 304 is located. In some examples, the location of the connection interfaces of the disposable probe 302 and the base 304 is within a predetermined distance (e.g., 0.5 inch, 1 inch, 1.5 inches, 2 inches, 3 inches, and so on) from both the end of the disposable probe 302 (that is opposite to the end on which the connection interface of the disposable probe 302 is located) and the end of the base 304 (that is opposite to the end of the base 304 on which the connection interface of the base 304 is located). That is, the location of the connection interfaces is equidistant from the end of the disposable probe 302 (that is opposite to the end on which the connection interface of the disposable probe 302 is located) and the end of the base 304 (that is opposite to the end of the base 304 on which the connection interface of the base 304 is located). In some examples, the location of the connection interfaces is closer to the end of the disposable probe 302 (that is opposite to the end on which the connection interface of the disposable probe 302 is located) than to the end of the base 304 (that is opposite to the end of the base 304 on which the connection interface of the base 304 is located).

In some arrangements, the disposable probe 202 and 302 are radially agnostic such that an operator can attach the disposable probe 202 or 302 to the base 204 or 304 without worrying about a radial orientation of the disposable probe 202 or 302, allowing for easy installation. In some arrangements, the disposable probe 202 or 302 reliably transmits all force, pressure, and/or torque into the base 204 or 304. In some arrangements, the frequency of operation for ultrasound transmission is determined by a capacitance. The capacitance may be laser trimmable one-time at a production facility. In some arrangements, the disposable probe 302 is configured as the base 204 and the base 304 is configured as the disposable probe 202.

In some examples, the disposable component (e.g., the disposable probes 101, 202, and 302) may include an enclosure (e.g., a baffle, cover, and so on) configured to cover any electronics, robotic components, wires, or other components of the base (e.g., the bases 102, 204, and 304) that may be exposed. In particular, the enclosure is configured to be attached to the base via suitable fasteners such as but not limited to, adhesives, hooks, latches, screws, press fittings, hook-and-loop fasteners, and so on.

The enclosure is configured to provide ingress protection against liquid (e.g., blood, sweat, and water) and particles (e.g., dust and hair) for the base and disposable probe. In some arrangements, the enclosure is made of an elastic material that can be form-fitted to at least a portion of the base when the enclosure is attached to the base. Furthermore, in some arrangements, the enclosure is made of a biocompatible material suitable for contacting a human body (e.g., the head of the subject). Moreover, in some arrangements, the enclosure is made of a lightweight material. Therefore, in considering ingress protection, elasticity, biocompatibility, and weight, the enclosure can be made from a material such as but not limited to, PE, PP, PC, PU, PEI, PVC, and PEEK in some examples. In other examples, the enclosure can be made from a layer of thin silicone, biocompatible waterproof fibers or fabric, a medical curtain (e.g., a PE curtain), treated paper, Tyvek®, and the like.

As such, the present disclosure is concerned with a method for providing a probe system (e.g., the probe systems 100, 200, and 300), the method including providing (e.g., manufacturing) a disposable component (e.g., the disposable probes 101, 202, and 302) and providing (e.g., manufacturing) a base (e.g., the bases 102, 204, and 304). Providing the base includes configuring or otherwise manufacturing the disposable component to be removably attached to the base.

In some arrangements, alternative to or in addition to the enclosure, a gasket (e.g., a silicone O-ring) may be disposed on the disposable component, the base, or both the disposable component and the base to seal any gap between the disposable component and the base when the disposable component is attached to the base. The gasket provides ingress protection for the electronics, robotic components, wires, or other components of the base that may be exposed by the gap between the disposable component and the base when the disposable component is attached to the base.

As shown in FIGS. 1-3F, when the disposable probe 101, 202, or 204 is removably attached to a respective one the base 102, 204, or 304, the disposable probe 101, 202, or 204 and the respective one the base 102, 204, or 304 form a unitary component that can move together as a single unit.

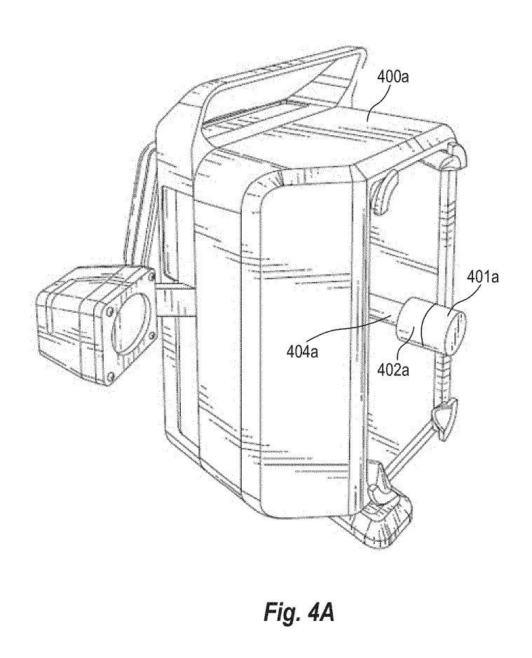

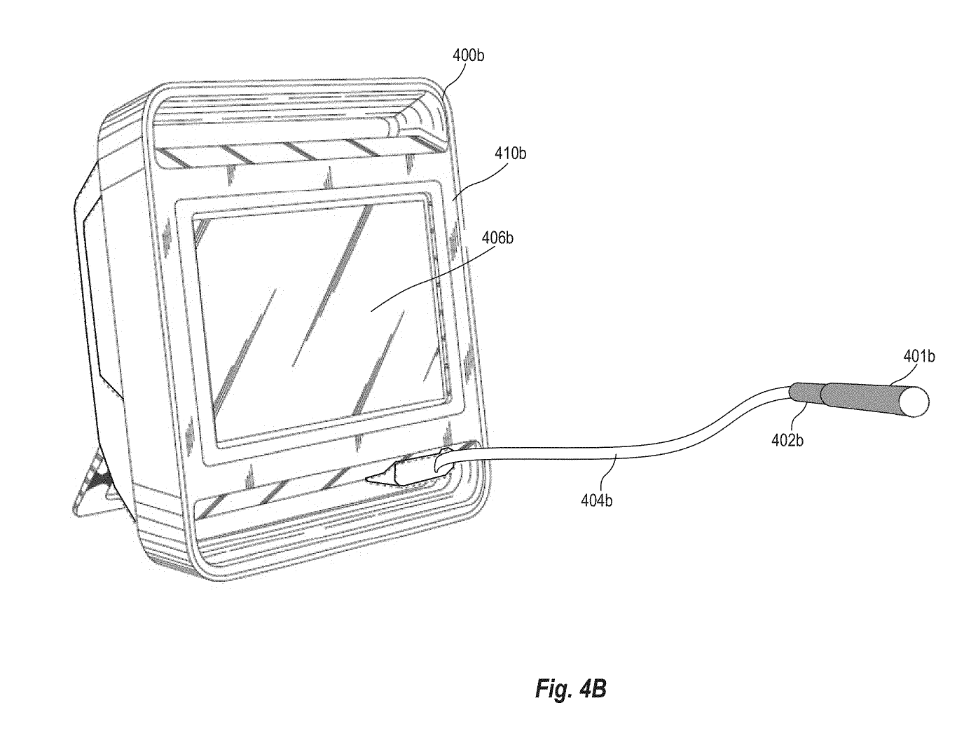

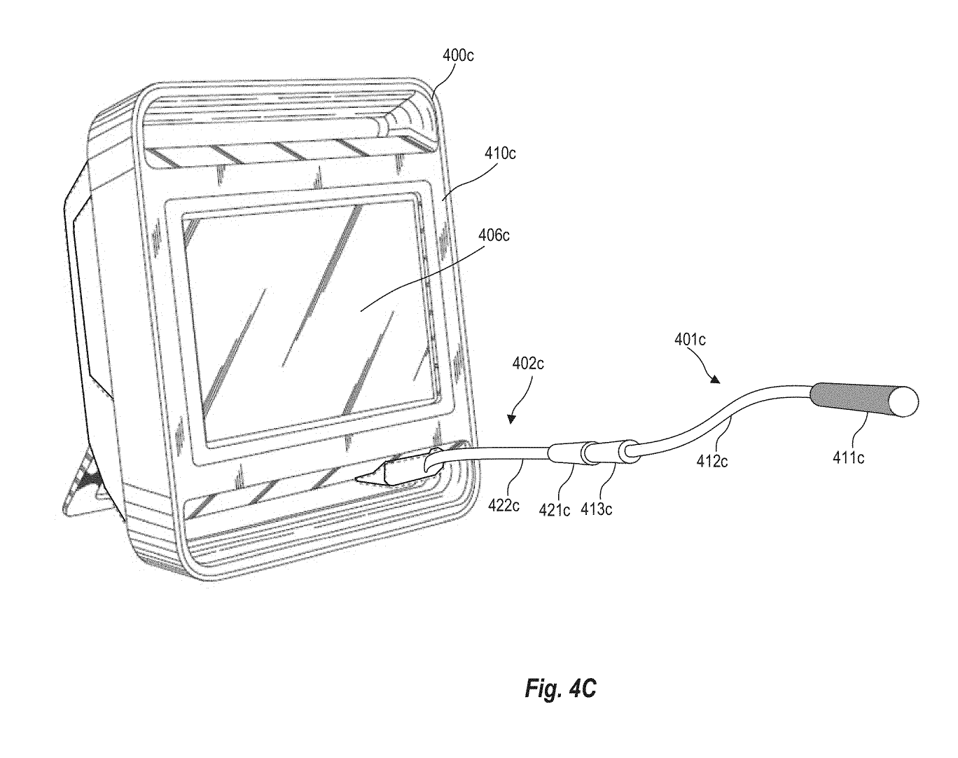

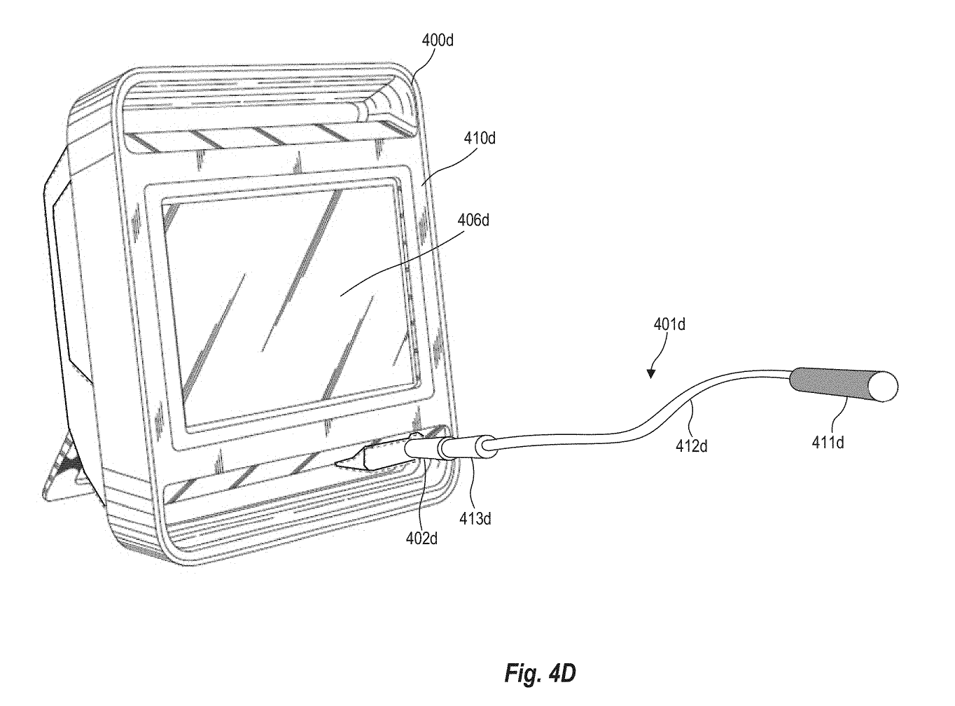

FIG. 4A shows an example implementation of the probe system 100, 200, or 300 in a system 400a according to various arrangements. Referring to FIGS. 1-4A, the system 400a includes a base 402a (e.g., the base 102, the base 204, or the base 304) that is removably attached and operatively coupled to a disposable probe 401a (e.g., the disposable probe 101, the disposable probe 202, or the disposable probe 302) in the manner described. In other words, the base 402a and the disposable probe 401a form a probe system such as but not limited to, the probe system 100, 200, or 300. The system 400a includes robotics (not shown) configured to automatically move the base 402a, and thus indirectly moving the disposable probe 401a when the disposable probe 401a is removably attached and operatively coupled to the base 402a. For example, the robotics (e.g., including components such as but not limited to, a motor assembly) are configured to translate the probe system (e.g., including the base 402a and the disposable probe 401a when the disposable probe 401a is attached to the base 402a) along a surface of the skin of the subject and to move the probe system toward and away from the head in the manner described. In some arrangements, a bottom end of the base 402a is coupled to the robotic system (e.g., to a support structure 404a movable by the motor assembly). The support structure 404a is shown to be a cylindrical rod that is coaxial with the disposable probe 401a and the base 402a. In some arrangements, the bottom end is fixed to the support structure 404a such that the base 402a and the support structure 404a form a unitary piece. In some arrangements, the bottom end of the base 402a includes a threaded section along a portion of the body of the base 402a, and the bottom end of the base 402a is secured to the support structure 404a via the threads (e.g., by being screwed into the support structure 404a). In other arrangements, the base 402a is secured to the support structure 404a by any other suitable connecting mechanisms such as but not limited to, welding, adhesive, one or more hooks and latches, one or more separate screws, press fittings, or the like. The base 402a and the disposable probe 401a can be removably coupled via the mechanisms described with respect to the probe system 100, 200, or 300.

When the disposable probe 401a is removably attached to the base 402a, the length of the probe system (including the length of the disposable probe 401a and the length of the base 402a) is less than a predetermined length (e.g., 3 inches, 5 inches, 7 inches, 8 inches, 10 inches, 12 inches, and so on). The location of the connection interfaces of the disposable probe 401a and the base 402a is within a predetermined distance (e.g., 0.5 inch, 1 inch, 1.5 inches, 2 inches, 3 inches, and so on) from an end of the disposable probe 401a opposite to the end on which the connection interface of the disposable probe 401a is located. The location of the connection interfaces of the disposable probe 401a and the base 402a is within a predetermined distance (e.g., 1 inch, 1.5 inches, 2 inches, 3 inches, 4 inches, and so on) from an end of the base 402a that is opposite to the end of the base 402a on which the connection interface of the base 402a is located. The support structure 404a is located on the end of the base 402a that is opposite to the end of the base 402a on which the connection interface of the base 402a is located. In some examples, the location of the connection interfaces of the disposable probe 401a and the base 402a is within a predetermined distance (e.g., 0.5 inch, 1 inch, 1.5 inches, 2 inches, 3 inches, and so on) from both the end of the disposable probe 401a (that is opposite to the end on which the connection interface of the disposable probe 401a is located) and the end of the base 402a (that is opposite to the end of the base 402a on which the connection interface of the base 402a is located). That is, the location of the connection interfaces is equidistant from the end of the disposable probe 401a (that is opposite to the end on which the connection interface of the disposable probe 401a is located) and the end of the base 402a (that is opposite to the end of the base 402a on which the connection interface of the base 402a is located). In some examples, the location of the connection interfaces is closer to the end of the disposable probe 401a (that is opposite to the end on which the connection interface of the disposable probe 401a is located) than to the end of the base 402a (that is opposite to the end of the base 402a on which the connection interface of the base 402a is located).

FIG. 4B shows an example implementation of the probe system 100, 200, or 300 in a system 400b according to various arrangements. Referring to FIGS. 1-4B, the system 400b includes a base 402b (e.g., the base 102, the base 204, or the base 304) that is removably attached and operatively coupled to a disposable probe 401b (e.g., the disposable probe 101, the disposable probe 202, or the disposable probe 302) in the manner described. In other words, the base 402b and the disposable probe 401b form a probe system such as but not limited to, the probe system 100, 200, or 300. The probe system as shown in FIG. 4B can be manually moved by an operator along a surface of the skin of the subject and toward and away from the head to collect data with respect to the subject. The base 402b and the disposable probe 401b can be removably coupled via the mechanisms described with respect to the probe system 100, 200, or 300. The base 402b is operatively coupled to a cable 404b. The base 402b can relay the signals received from the disposable probe 401b and/or send data obtained by the base 402b processing the signals received from the disposable probe 401b to a processing unit of the system 400b (housed within a housing 410b of the system 400b) for further processing or to a display 406b of the system 400b for displaying. The cable 404b is shown to be coaxial with the disposable probe 401b and the base 402b. In some arrangements, the bottom end of the base 402b is fixed to the cable 404b such that the base 402b and the cable 404b form a unitary piece. In some arrangements, the bottom end of the base 402b includes a threaded section along a portion of the body of the base 402b, and the bottom end of the base 402b is secured to the cable 404b via the threads (e.g., by being screwed into the cable 404b). In other arrangements, the base 402b is secured to the cable 404b by any other suitable connecting mechanisms such as but not limited to, welding, adhesive, one or more hooks and latches, one or more separate screws, press fittings, or the like.

When the disposable probe 401b is removably attached to the base 402b, the length of the probe system (including the length of the disposable probe 401b and the length of the base 402b) is less than a predetermined length (e.g., 3 inches, 5 inches, 7 inches, 8 inches, 10 inches, 12 inches, and so on). The location of the connection interfaces of the disposable probe 401b and the base 402b is within a predetermined distance (e.g., 0.5 inch, 1 inch, 1.5 inches, 2 inches, 3 inches, and so on) from an end of the disposable probe 401b opposite to the end on which the connection interface of the disposable probe 401b is located. The location of the connection interfaces of the disposable probe 401b and the base 402b is within a predetermined distance (e.g., 1 inch, 1.5 inches, 2 inches, 3 inches, 4 inches, and so on) from an end of the base 402b that is opposite to the end of the base 402b on which the connection interface of the base 402b is located. The cable 404b is located on the end of the base 402b that is opposite to the end of the base 402b on which the connection interface of the base 402b is located. In some examples, the location of the connection interfaces of the disposable probe 401b and the base 402b is within a predetermined distance (e.g., 0.5 inch, 1 inch, 1.5 inches, 2 inches, 3 inches, and so on) from both the end of the disposable probe 401b (that is opposite to the end on which the connection interface of the disposable probe 401b is located) and the end of the base 402b (that is opposite to the end of the base 402b on which the connection interface of the base 402b is located). That is, the location of the connection interfaces is equidistant from the end of the disposable probe 401b (that is opposite to the end on which the connection interface of the disposable probe 401b is located) and the end of the base 402b (that is opposite to the end of the base 402b on which the connection interface of the base 402b is located). In some examples, the location of the connection interfaces is closer to the end of the disposable probe 401b (that is opposite to the end on which the connection interface of the disposable probe 401b is located) than to the end of the base 402b (that is opposite to the end of the base 402b on which the connection interface of the base 402b is located).

FIG. 4C shows an example implementation of the probe system 100, 200, or 300 in a system 400c according to various arrangements. Referring to FIGS. 1-4C, the system 400c includes a base portion 402c that is removably attached and operatively coupled to a disposable portion 401c. The disposable portion 401c includes an instrument 411c, a cable 412c, and a connection interface 413c. The instrument 411c includes devices/instruments configured to collect data (e.g., ultrasound data, sound data, light data, electromagnetic data, temperature data, pressure data, and so on) with respect to a subject and transmit signals corresponding to the data to the connection interface 413c via the cable 412c. In one example, the instrument 411c includes one or more of the components 120-128, 130-136, and 140 of the disposable probe 101. The instrument 411c can be manually moved by an operator along a surface of the skin of the subject and toward and away from the head to collect data with respect to the subject. The connection interface 413c may be a connection interface such as but not limited to, the connection interface 150 of the disposable probe 101, the connection interface of the disposable probe 202, or the connection interface of the disposable probe 302. The disposable portion 401c is disposable. The connection interface 413c is configured to be removably attached to a connection interface 421c of the base portion 402c. The base portion 402c further includes a cable 422c. The connection interface 421c transmits the signals received from the connection interface 413c to a processing unit of the system 400c (housed within a housing 410c of the system 400c) for further processing or to a display 406c of the system 400c for displaying. The connection interface 421c may be a connection interface such as but not limited to, the connection interface 155 of the base 102, the connection interface of the base 204, or the connection interface of the base 304. When the disposable portion 401c (e.g., the connection interface 413c) is removably attached to the connection interface 421c of the base portion 402c, the length of the probe system (including the length of the instrument 411c, the length of the cable 412c when the cable 412c is fully extended, the length of the connection interfaces 413c and 421c) is less than a predetermined length (e.g., 5 inches, 7 inches, 8 inches, 10 inches, 12 inches, 15 inches, 20 inches, and so on). The location of where the connection interfaces 413c and 421c connect is within a predetermined distance (e.g., 3 inches, 5 inches, 6 inches, 8 inches, 10 inches, 13 inches, 18 inches, and so on) from an end of the instrument 411c opposite to the end on which the cable 412c is connected when the cable 412c is fully extended. The location of where the connection interfaces 413c and 421c connect is within a predetermined distance (e.g., 1 inch, 1.5 inches, 2 inches, 3 inches, 4 inches, and so on) from an end of the connection interface 421c that connects to the cable 422c. In some examples, the location of where the connection interfaces 413c and 421c connect is within a predetermined distance (e.g., 3 inches, 4 inches, 7 inches 10 inches, 13 inches, 18 inches, and so on) from both the end of the instrument 411c (opposite to the end on which the cable 412c is connected) when the cable 412c is fully extended and the end of the connection interface 421c (that connects to the cable 422c). That is, the location of where the connection interfaces 413c and 421c connect is equidistant from the end of the instrument 411c (opposite to the end on which the cable 412c is connected) when the cable 412c is fully extended and the end of the connection interface 421c (that connects to the cable 422c). In some examples, the location of where the connection interfaces 413c and 421c connect is closer to the end of the connection interface 421c (that connects to the cable 422c) than the end of the instrument 411c (opposite to the end on which the cable 412c is connected) when the cable 412c is fully extended.

FIG. 4D shows an example implementation of the probe system 100, 200, or 300 in a system 400d according to various arrangements. Referring to FIGS. 1-4D, the system 400d includes a base 402d that is removably attached and operatively coupled to a disposable portion 401d. The disposable portion 401d includes an instrument 411d, a cable 412d, and a connection interface 413d. The instrument 411d includes devices/instruments configured to collect data (e.g., ultrasound data, sound data, light data, electromagnetic data, temperature data, pressure data, and so on) with respect to a subject and transmit signals corresponding to the data to the connection interface 413d via the cable 412d. In one example, the instrument 411d includes one or more of the components 120-128, 130-136, and 140 of the disposable probe 101. The instrument 411d can be manually moved by an operator along a surface of the skin of the subject and toward and away from the head to collect data with respect to the subject. The connection interface 413d may be a connection interface such as but not limited to, the connection interface 150 of the disposable probe 101, the connection interface of the disposable probe 202, or the connection interface of the disposable probe 302. The disposable portion 401d is disposable. The connection interface 413d is configured to be removably attached to a connection interface of the base 402d. The base 402d transmits the signals received from the connection interface 413d to a processing unit of the system 400d (housed within a housing 410d of the system 400d) for further processing or to a display 406d of the system 400d for displaying. The connection interface of the base 402d may be a connection interface such as but not limited to, the connection interface 155 of the base 102, the connection interface of the base 204, or the connection interface of the base 304. The base 402d may be a base such as but not limited to, the base 102, the base 204, or the base 304.

When the disposable portion 401d (e.g., the connection interface 413d) is removably attached to the base 402d, the length of the probe system (including the length of the instrument 411d, the length of the cable 412d when the cable 412d is fully extended, the length of the connection interface 413d and the base 402d) is less than a predetermined length (e.g., 5 inches, 7 inches, 8 inches, 10 inches, 12 inches, 15 inches, 20 inches, and so on). The location of where the connection interface 413d connects to the base 402d is within a predetermined distance (e.g., 3 inches, 5 inches, 6 inches, 8 inches, 10 inches, 13 inches, 18 inches, and so on) from an end of the instrument 411d opposite to the end on which the cable 412d is connected when the cable 412d is fully extended. The location of where the connection interface 413d connects to the base 402d is within a predetermined distance (e.g., 1 inch, 1.5 inches, 2 inches, 3 inches, 4 inches, and so on) from an end of the base 402d that connects to the housing 410d. In some examples, the location of where the connection interface 413d connects to the base 402d is within a predetermined distance (e.g., 3 inches, 4 inches, 7 inches 10 inches, 13 inches, 18 inches, and so on) from both the end of the instrument 411d (opposite to the end on which the cable 412d is connected) when the cable 412d is fully extended and the end of the base 402d (that connects to the housing 410d). That is, the location of where the connection interface 413d and the base 402d connect is equidistant from the end of the instrument 411d (opposite to the end on which the cable 412d is connected) when the cable 412d is fully extended and the end of the base 402d (that connects to the housing 410d). In some examples, the location of where the connection interface 413d connects the base 402d is closer to the end of the base 402d (that connects to the housing 410d) than the end of the instrument 411d (opposite to the end on which the cable 412d is connected) when the cable 412d is fully extended.

The above used terms, including “held fast,” “mount,” “attached,” “coupled,” “affixed,” “connected,” “secured,” and the like are used interchangeably. In addition, while certain arrangements have been described to include a first element as being “coupled” (or “attached,” “connected,” “fastened,” etc.) to a second element, the first element may be directly coupled to the second element or may be indirectly coupled to the second element via a third element.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout the previous description that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

It is understood that the specific order or hierarchy of blocks in the processes disclosed is an example of illustrative approaches. Based upon design preferences, it is understood that the specific order or hierarchy of blocks in the processes may be rearranged while remaining within the scope of the previous description. The accompanying method claims present elements of the various blocks in a sample order, and are not meant to be limited to the specific order or hierarchy presented.

The previous description of the disclosed implementations is provided to enable any person skilled in the art to make or use the disclosed subject matter. Various modifications to these implementations will be readily apparent to those skilled in the art, and the generic principles defined herein may be applied to other implementations without departing from the spirit or scope of the previous description. Thus, the previous description is not intended to be limited to the implementations shown herein but is to be accorded the widest scope consistent with the principles and novel features disclosed herein.

The various examples illustrated and described are provided merely as examples to illustrate various features of the claims. However, features shown and described with respect to any given example are not necessarily limited to the associated example and may be used or combined with other examples that are shown and described. Further, the claims are not intended to be limited by any one example.

The foregoing method descriptions and the process flow diagrams are provided merely as illustrative examples and are not intended to require or imply that the blocks of various examples must be performed in the order presented. As will be appreciated by one of skill in the art the order of blocks in the foregoing examples may be performed in any order. Words such as “thereafter,” “then,” “next,” etc. are not intended to limit the order of the blocks; these words are simply used to guide the reader through the description of the methods. Further, any reference to claim elements in the singular, for example, using the articles “a,” “an” or “the” is not to be construed as limiting the element to the singular.

The various illustrative logical blocks, modules, circuits, and algorithm blocks described in connection with the examples disclosed herein may be implemented as electronic hardware, computer software, or combinations of both. To clearly illustrate this interchangeability of hardware and software, various illustrative components, blocks, modules, circuits, and blocks have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The preceding description of the disclosed examples is provided to enable any person skilled in the art to make or use the present disclosure. Various modifications to these examples will be readily apparent to those skilled in the art, and the generic principles defined herein may be applied to some examples without departing from the spirit or scope of the disclosure. Thus, the present disclosure is not intended to be limited to the examples shown herein but is to be accorded the widest scope consistent with the following claims and the principles and novel features disclosed herein.

It should be noted that although the diagrams herein may show a specific order and composition of method blocks, it is understood that the order of these blocks may differ from what is depicted. For example, two or more blocks may be performed concurrently or with partial concurrence. Also, some method blocks that are performed as discrete blocks may be combined, blocks being performed as a combined block may be separated into discrete blocks, the sequence of certain processes may be reversed or otherwise varied, and the nature or number of discrete processes may be altered or varied. The order or sequence of any element or apparatus may be varied or substituted according to alternative arrangements. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web arrangements of the present disclosure could be accomplished with standard programming techniques with rule based logic and other logic to accomplish the various database searching blocks, correlation blocks, comparison blocks, and decision blocks.

What is claimed is:

1. A disposable probe system for use in an ultrasound system, comprising:
a disposable probe configured to transmit or receive acoustic energy; and
a base included in the ultrasound system, wherein the disposable probe is configured to be removably attached to the base.

2. The disposable probe system of claim 1, wherein
the disposable probe has a first connection interface;
the base has a second connection interface; and
the disposable probe is configured to be removably attached to the base by having the first connection interface removably attach to the second connection interface.

3. The disposable probe system of claim 2, wherein the first connection interface is configured to be attached to the second connection interface and detached from the second connection interface without using tools.

4. The disposable probe system of claim 2, wherein
the first connection interface comprises a pin;
the second connection interface comprises a recessed contact configured to receive the pin; and
the first connection interface is configured to be attached to the second connection interface when the pin is received by the recessed contact.

5. The disposable probe system of claim 4, wherein
an electrical connection is closed when the pin is received by the recessed contact;
the electrical connection is configured to allow transmission of data or power between the first connection interface and the second connection interface.

6. The disposable probe system of claim 2, wherein
the first connection interface comprises a metal contact;
the second connection interface comprises a magnet configured to be coupled to the metal contact; and
the first connection interface is configured to be attached to the second connection interface when the magnet is magnetically attracted to the metal contact.

7. The disposable probe system of claim 2, wherein
the first connection interface is configured to be within a predetermined distance from a scanning surface; and
the disposable probe is configured to transmit or receive the acoustic energy with respect to the scanning surface.

8. The disposable probe system of claim 1, further includes a robotic system, wherein
the base is configured to be coupled to the robotic system; and
the robotic system is configured to move the disposable probe by moving the base when the disposable probe is attached to the base.

9. The disposable probe system of claim 1, wherein the disposable probe is configured to be manually operated.

10. The disposable probe system of claim 1, wherein the disposable probe comprises an ultrasound instrument configured to generate ultrasound energy.

11. The disposable probe system of claim 10, wherein the ultrasound instrument comprises at least one of a coupling layer, a first metallic layer, a lead zirconate titanate (PZT) layer, a second metallic layer, or a circuit board.

12. The disposable probe system of claim 10, wherein the disposable probe comprises a concave surface configured to focus the ultrasound energy.

13. The disposable probe system of claim 12, further comprising a membrane containing gel.

14. The disposable probe system of claim 13, wherein the membrane is coated with a lubricant.

15. The disposable probe system of claim 1, further comprising a force sensor between the disposable probe and the base.

16. The disposable probe system of claim 1, wherein
the disposable probe comprises a jack;
the base comprises a cavity; and
the disposable probe is configured to be attached to the base when the jack is inserted into the cavity.

17. The disposable probe system of claim 16, wherein
the jack comprises at least one section;
the base comprises at least one electrical contact;
each of the at least one section is configured to electrically couple to a corresponding one of the at least one electrical contact when the jack is inserted into the cavity.

18. The disposable probe system of claim 1, wherein the disposable probe comprises at least one of a pressure sensor or a temperature sensor.

19. The disposable probe system of claim 1, wherein the disposable probe comprises one or more light emitting diodes (LEDs).

20. The disposable probe system of claim 1, wherein the disposable probe comprises one or more liquid-crystal displays (LCDs),

21. The disposable probe system of claim 1, wherein the disposable probe is mechanically keyed to the base.

22. The disposable probe system of claim 1, wherein
the disposable probe comprises a memory; and
the disposable probe is keyed to the base via at least one identifier stored in the memory.

23. The disposable probe system of claim 1, wherein the disposable probe and the base form a unitary component when the disposable probe is removably attached to the base.

24. A method for providing a disposable probe system for use in an ultrasound system, comprising:
providing a disposable probe configured to transmit or receive acoustic energy; and
providing a base included in the ultrasound system, wherein providing the base comprises configuring the disposable probe to be removably attached to the base.
